(19) United States
(12) Patent Application Publication
(10) Pub. No.: US 2026/0327603 A1
(43) Pub. Date: Oct. 22, 2026

(54) PREPARATION OF LIBRARIES OF BISPECIFIC BINDERS EXPRESSED IN EUKARYOTIC CELLS

(71) Applicant: Iontas Limited, London (GB)

(72) Inventors: Kothai Nachiar Devi PARTHIBAN, Cambridge (GB); Peter SLAVNY, Cambridge (GB); Cyril V. PRIVEZENTZEV, Cambridge (GB)

(21) Appl. No.: 19/486,480

(22) PCT Filed: Jun. 5, 2024
(86) PCT No.: PCT/EP2024/065476

§ 371 Date: Nov. 21, 2025

(30) Foreign Application Priority Data
Jun. 6, 2023 (EP) 23177536.2

(51) Int. Cl. C12N 15/10; C07K 16/00; C12N 15/62

(52) U.S. Cl. CPC C12N 15/1037; C07K 16/005; C12N 15/62; C07K 2317/31; C07K 2317/569; C07K 2317/622

(57) ABSTRACT

Preparation of libraries of bispecific binders expressed in eukaryotic cells Described herein are methods for producing a library of eukaryotic cell clones containing DNA encoding a diverse repertoire of bispecific binders, in particular for bispecific binders comprising a first binding domain coupled to a first Fc domain and a second binding domain coupled to a second Fc domain.

### FIELD

Aspects and embodiments described herein relate to the field of production of libraries of eukaryotic cell clones, specifically to libraries of eukaryotic cell clones containing DNA encoding a diverse repertoire of bispecific binders.

### BACKGROUND

Cancers and other complex diseases are often underlied by various factors and multiple signaling pathways. Targeting more than one molecule can be useful to circumvent ‘escape’ effects and to avoid treatment resistance observed with monospecific antibodies. Bispecific antibodies have the potential to overcome these drawbacks, but their success has been hindered by the complexities associated with generating appropriate molecules for both research- and large-scale manufacturing purposes and by the lack of suitable libraries of bispecific antibodies for functional screening. Indeed: while over 100 different bispecific antibody formats have been described, a reliable approach for the generation of diverse, high-affinity antibody libraries has not been shown for most of them. One of the major hurdles is related to the difficulties associated with achieving successful expression of correctly formatted bispecific antibodies. Depending on the bispecific antibody format, optimization is needed to have both bispecific modules selected to achieve the proper folding of both modules. In particular, the expressed bispecific antibody must still be fully functional, i.e. able to simultaneously bind its two antigens. The functional activity of bispecifics, particularly for bispecific antibodies that rely on the simultaneous binding to 2 different targets, is poorly predictable from that of the individual parental antibodies.

Currently available systems typically provide individual antibody repertoires to one target and do not allow to combine them in the final desired bispecific format. It is not obvious that when monospecific antibody repertoires to two different targets or two different epitopes on the same target are brought together in a particular antibody format the corresponding bispecific molecules or biparatopic molecules show simultaneous binding. Reasons could be related to (1) steric hindrance of binding unit 1 on binding unit 2 to the target(s) or visa versa or (2) interference of both antibody binding domains on each other disrupting the bispecific binding nature. The methods described in this application provides a solution towards these problems.

The methods described herein allow display of bi- and multispecific antibodies in the final desired (therapeutic) format using mammalian display avoiding the need to investigate all individual members of such a bispecific antibody.

WO2015/166272 describes a method of producing a library of eukaryotic cell clones containing DNA encoding a diverse repertoire of binders. WO2015/166272 does not relate specifically to bispecific antibodies and does not disclose a method for producing libraries of bispecific antibodies, especially not for bispecific antibodies comprising a first binding domain coupled to a first Fc domain and a second binding domain coupled to a second Fc domain.

### SUMMARY

In view of the above, there is still a need for methods of producing a library of eukaryotic cell clones containing DNA encoding a diverse repertoire of bispecific binders in the final desired (therapeutic) format.

It has surprisingly been found that the methods disclosed herein allow for eukaryotic cells to successfully express heterodimeric bispecific antibodies and anchor them to the cell surface, in particular for bispecific antibodies comprising a first binding domain coupled to a first Fc domain and a second binding domain coupled to a second Fc domain. Particularly, as elaborated elsewhere herein and in the experimental part, it has been found that bispecific antibodies, particularly bispecific antibodies comprising a first binding domain coupled to a first Fc domain and a second binding domain coupled to a second Fc domain, can be successfully displayed while retaining their full functionality of binding to two separate antigens (i.e., achieving proper expression and display on the cell surface, achieving proper folding of both binding domains, and avoiding steric hindrance or interference between both variable domains). Therefore, the methods disclosed herein make possible the construction of bispecific mammalian display libraries.

In an aspect, there is provided a method for producing a library of eukaryotic cell clones containing DNA encoding a diverse repertoire of bispecific binders, the method comprising:
providing eukaryotic cells;
providing a plurality of donor DNA molecules, each donor DNA molecule comprising a first nucleic acid sequence encoding a first binding domain coupled to a first Fc domain; and a second nucleic acid sequence encoding a second binding domain coupled to a second Fc domain;
introducing the donor DNA into the cells; and
culturing the recombinant cells to produce clones, thereby providing a library of eukaryotic cell clones containing DNA encoding the repertoire of bispecific binders.

Preferably, the first and second Fc domains are engineered to promote heterodimerization. Accordingly, there is provided a method for producing a library of eukaryotic cell clones containing DNA encoding a diverse repertoire of bispecific binders, the method comprising:
providing eukaryotic cells;
providing a plurality of donor DNA molecules, each donor DNA molecule comprising a first nucleic acid sequence encoding a first binding domain coupled to a first Fc domain; and a second nucleic acid sequence encoding a second binding domain coupled to a second Fc domain, wherein the first and second Fc domains are engineered to promote heterodimerization;
introducing the donor DNA into the cells; and
culturing the recombinant cells to produce clones, thereby providing a library of eukaryotic cell clones containing DNA encoding the repertoire of bispecific binders.

A method according to this aspect may be called “a method for producing a library as described herein” or “a method for producing a library” or the like in the context of this disclosure.

In some embodiments, a method for producing a library as described herein is such that the first and/or second binding domain comprises a single antibody variable domain, preferably a variable domain derived from a heavy-chain antibody such as a VHH or a V_{NAR}, and/or wherein the first and/or second binding domain comprises two antibody variable domains, preferably a single-chain variable fragment (scFv). In some embodiments, the first binding domain comprises a single antibody variable domain, preferably a variable domain derived from a heavy-chain antibody such as a VHH or a V_{NAR}, more preferably a VHH. In some embodiments, the second binding domain comprises two antibody variable domains, preferably a single-chain variable fragment (scFv).

In some embodiments, a method for producing a library as described herein is such that the first and second Fc domain are engineered to promote heterodimerization, preferably wherein the first Fc domain comprises a knob mutation and the second Fc domain comprises a hole mutation, or wherein the first Fc domain comprises a hole mutation and the second Fc domain comprises a knob mutation.

Bispecific binders according to this disclosure are preferably multimeric binders comprising at least a first and second subunit (i.e., separate polypeptide chains). More preferably, bispecific binders according to this disclosure are dimeric binders. Multimeric binders including dimeric binders may be obtained by expression and assembly of the separately encoded subunits. The multiple subunits may be encoded on the same molecule of donor DNA, as described above. However, it may also be desirable to integrate the different subunits into separate loci, in which case the subunits can be provided on separate donor DNA molecules. These could be integrated within the same step of introducing the donor DNA into the cells or they may be integrated sequentially.

In an aspect, methods for producing libraries of eukaryotic cell clones containing DNA encoding a diverse repertoire of multimeric, e.g. dimeric, bispecific binders may comprise:
providing eukaryotic cells containing DNA encoding a first subunit of the bispecific binders, the DNA comprising a first nucleic acid sequence encoding a first binding domain coupled to a first Fc domain;
providing a plurality of donor DNA molecules encoding a second subunit of the bispecific binders, each donor DNA molecule comprising a second nucleic acid sequence encoding a second binding domain coupled to a second Fc domain;
introducing the donor DNA into the cells, thereby creating recombinant cells which contain donor DNA integrated in the cellular DNA; and
culturing the recombinant cells to produce clones containing DNA encoding the first and second subunits, thereby providing a library of eukaryotic cell clones containing DNA encoding the repertoire of bispecific binders.

In the above example, donor DNA encoding a second subunit is introduced into cells already containing DNA encoding a first subunit. An alternative approach is to integrate a first subunit in a first cycle of introducing donor DNA, followed by introducing the second subunit in a second cycle of introducing donor DNA.

Thus, in an aspect, methods for producing libraries of eukaryotic cell clones containing DNA encoding a diverse repertoire of multimeric, e.g. dimeric, bispecific binders may comprise:
providing eukaryotic cells;
providing a plurality of first donor DNA molecules encoding a first subunit of the bispecific binders, each donor DNA molecule comprising a first nucleic acid sequence encoding a first binding domain coupled to a first Fc domain;
introducing the first donor DNA into the cells to create a first set of recombinant cells containing the first donor DNA integrated in the cellular DNA;
culturing the first set of recombinant cells to produce a first set of clones containing DNA encoding the first subunit;
providing a plurality of second donor DNA molecules encoding a second subunit of the bispecific binders, each donor DNA molecule comprising a second nucleic acid sequence encoding a second binding domain coupled to a second Fc domain;
introducing the second donor DNA into cells of the first set of clones to create a second set of recombinant cells containing the first and second donor DNA integrated in the cellular DNA; and
culturing the second set of recombinant cells to produce a second set of clones containing DNA encoding the first and second subunits, thereby providing a library of eukaryotic cell clones containing DNA encoding the repertoire of bispecific binders.

Also, in another aspect, methods for producing libraries of eukaryotic cell clones containing DNA encoding a diverse repertoire of multimeric, e.g. dimeric, bispecific binders may comprise:
providing eukaryotic cells;
providing a plurality of first donor DNA molecules encoding a first subunit of the bispecific binders, each donor DNA molecule comprising a first nucleic acid sequence encoding a first binding domain coupled to a first Fc domain;
providing a plurality of second donor DNA molecules encoding a second subunit of the bispecific binders, each donor DNA molecule comprising a second nucleic acid sequence encoding a second binding domain coupled to a second Fc domain;
introducing the first and second donor DNA into the cells to create recombinant cells containing the first and second donor DNA integrated in the cellular DNA; and
culturing the recombinant cells to produce clones, thereby providing a library of eukaryotic cell clones containing DNA encoding the repertoire of bispecific antibodies.

“A method as described herein” and similar expressions as used herein refer to any of the above methods for producing libraries of eukaryotic cell clones encoding a diverse repertoire of (multimeric, including dimeric) bispecific binders.

Methods as described herein such as methods for generating a library may involve providing a site-specific nuclease which cleaves a recognition sequence in cellular DNA. More particularly, in some embodiments in the context of methods as described herein, the step of introducing donor DNA into cells comprises providing a site-specific nuclease within the cells, wherein the nuclease cleaves a recognition sequence in cellular DNA to create an integration site at which the donor DNA becomes integrated into the cellular DNA, integration occurring through DNA repair mechanisms endogenous to the cells, thereby creating recombinant cells containing donor DNA integrated in the cellular DNA. In some embodiments, the donor DNA molecules are flanked by homology arms. This may increase the integration efficiency. In some embodiments, the donor DNA molecules comprise a first promoter operably linked to the first nucleic acid sequence and/or the donor DNA molecules comprise a second promoter operably linked to the second nucleic acid sequence. In some embodiments, the donor DNA molecules comprise a bidirectional promoter operably linked to the first and second nucleic acid sequence. In some embodiments, the donor DNA molecules comprise a bidirectional promoter operably linked to the first and second nucleic acid sequence. In some embodiments, the first and/or second nucleic acid sequence encodes a membrane anchor, such as a transmembrane domain or a membrane localization signal. In some embodiments, the eukaryotic cells are higher eukaryotic cells with a genome size of greater than 2×10⁷ base pairs, preferably mammalian, avian, insect or plant cells, more preferably mammalian cells.

Integration of donor DNA into cellular DNA creates recombinant cells, which can be cultured to produce clones. Individual recombinant cells into which the donor DNA has been integrated are thus replicated to generate clonal populations of cells—“clones”—each clone being derived from one original recombinant cell. Thus, the method generates a number of clones corresponding to the number of cells into which the donor DNA was successfully integrated. The collection of clones form a library encoding the repertoire of bispecific binders (or, at an intermediate stage where bispecific binder subunits are integrated in separate rounds, the clones may encode a set of bispecific binder subunits). Methods as described herein can thus provide a library of eukaryotic cell clones containing DNA encoding the repertoire of bispecific binders.

Accordingly, in an aspect, there is provided a library of eukaryotic cell clones containing DNA encoding a diverse repertoire of bispecific binders, wherein the library is obtained via a method for generating a library as described herein. Such libraries according to this aspect may be called a “library as described herein” or the like in the context of this application.

Methods as described herein can generate libraries of eukaryotic cell clones that express a diverse repertoire of bispecific binders, each cell containing recombinant DNA wherein donor DNA encoding a bispecific binder or subunit of a bispecific binder is integrated. The donor DNA may be integrated at a fixed locus, or optionally at multiple fixed loci, in the cellular DNA. By “fixed” it is meant that the locus is the same between cells. Cells used for creation of the library may therefore contain a nuclease recognition sequence at a fixed locus, representing a universal landing site in the cellular DNA at which the donor DNA can integrate. The recognition sequence for the site-specific nuclease may be present at one or more than one position in the cellular DNA. Accordingly, in an aspect, there is provided an in vitro library of eukaryotic cell clones that express a diverse repertoire of at least 10\hat{ }3, 10\hat{ }4, 10\hat{ }5, 10\hat{ }6, 10\hat{ }7, 10\hat{ }8 or 10\hat{ }9 different bispecific binders, each cell containing recombinant DNA wherein donor DNA encoding a bispecific binder or subunit of a bispecific binder is integrated in at least a first and/or a second locus in the cellular DNA; optionally wherein the locus is a fixed locus. A “library as described herein” or the like as used herein also refers to such an in vitro library of eukaryotic cell clones.

Libraries produced according to the methods described herein may be employed in a variety of ways. A library may be cultured to express the bispecific binders, thereby producing a diverse repertoire of bispecific binders. A library may be used for screening for cells displaying a desired phenotype, wherein the phenotype results from expression of a bispecific binder by a cell. Accordingly, in an aspect, there is provided a method of screening for a cell of a desired phenotype, wherein the phenotype results from expression of a bispecific binder by the cell, the method comprising:
providing a library via the method for producing a library as described herein, or
providing a library as described herein;
culturing the library cells to express the bispecific binders; and
detecting whether the desired phenotype is exhibited.

A method according to this aspect may be called “a method of screening for a cell of a desired phenotype as described herein” or the like. “A method as described herein” and the like as used herein also refers to the above method of screening for a cell of a desired phenotype.

Phenotype screening is possible in which library cells are cultured to express the bispecific binders, followed by detecting whether the desired phenotype is exhibited in clones of the library. Cellular read-outs can be based on alteration in cell behaviour such as altered expression of endogenous or exogenous reporter genes, differentiation status, proliferation, survival, cell size, metabolism or altered interactions with other cells. When the desired phenotype is detected, cells of a clone that exhibits the desired phenotype may then be recovered. Optionally, DNA encoding the bispecific binder is then isolated from the recovered clone, providing DNA encoding a bispecific binder which produces the desired phenotype when expressed in the cell. Optionally, the DNA encoding a bispecific binder which produces the desired phenotype may be sequenced.

A library may also be used for screening to identify bispecific binders that recognise a target of interest, optionally two targets of interest. Accordingly, in an aspect, there is provided a method for screening to identify bispecific binders to a target of interest, optionally two targets of interest, said method comprising:
providing a library via the method for producing a library as described herein, or
providing a library as described herein;
culturing cells of the library to express the bispecific binders;
exposing the bispecific binders to the target, optionally to the two targets, allowing recognition of the target, optionally of the two targets, by one or more binders of interest, if present; and
detecting whether the target, optionally the two targets, is recognised by a binder of interest.

A method according to this aspect may be called “a method for screening to identify a binder to a target of interest as described herein” or “a method for screening to identify a binder” or the like. “A method as described herein” as used herein also refers to the above method for screening to identify a binder to a target of interest.

In such methods a library is cultured to express the bispecific binders, and the bispecific binders are exposed to the target(s) to allow recognition of the target(s) by one or more binders of interest, if present, and detecting whether the target(s) is recognised by a binder of interest. When the desired target(s) is bound, cells of a clone containing DNA encoding the binder of interest may then be recovered. Optionally, DNA encoding the bispecific binder is then isolated from the recovered clone, providing DNA encoding a bispecific binder that binds the target(s) of interest. Optionally, the DNA encoding a binder that recognises the target may be sequenced.

In such methods, bispecific binders may be displayed on the cell surface and those clones of the library that display bispecific binders with desired properties can be isolated. Thus, cells incorporating genes encoding bispecific binders with desired functional or binding characteristics could be identified within the library. The genes can be recovered and used for production of the bispecific binder or used for further engineering to create derivative libraries of bispecific binders to yield bispecific binders with improved properties.

In an aspect, there is provided a bispecific binder that has been identified from a library as described herein, for example a bispecific binder that was identified using a method for screening to identify a bispecific binder to a target of interest as described herein. Preferred bispecific binders are described elsewhere herein.

Various features of the aspects and embodiments of this disclosure are further described below. It is noted that headings used throughout this specification are to assist navigation only and should not be interpreted as definitive, and that features described in different sections may be relevant for all aspects and embodiments described herein and may thus be combined as appropriate.

### DETAILED DESCRIPTION

### Bispecific Binders

A “binder” as described herein is a binding molecule, representing a specific binding partner for another molecule. Typical examples of specific binding partners are antibody-antigen and receptor-ligand. Preferably, a (bispecific) binder as described herein is a (bispecific) antibody.

A “bispecific binder” as used herein denotes a molecule comprising binding domains for two different antigens or two different epitopes on the same antigen. A bispecific binder is composed of two “monospecific” subunits which can be brought together (heterodimerized) in a bispecific format, hence forming a “bispecific binder”. Accordingly, throughout this disclosure, references to a “bispecific binder” may be replaced with references to “subunits of a bispecific binder”. The subunits are typically engineered to promote the formation of the bispecific/heterodimeric format, for example by using Fc domains engineered to promote heterodimerization. Libraries of cell clones encoding a repertoire of such bispecific binders allow the identification of optimal bispecific binders directly in the final (therapeutic) format.

The repertoire of bispecific binders encoded by a library will usually share a common structure (constant domain, e.g. Fc domain) and have one or more regions of diversity (variable domain). The library therefore enables selection of a member of a desired structural class of molecules. For example, the bispecific binders may be polypeptides sharing a common structure (constant domain, e.g. Fc domain) and having one or more regions of amino acid sequence diversity (variable domain).

This can be illustrated by considering a repertoire of bispecific antibody molecules. These may be antibody molecules of a common structural class, e.g., scFv-Fc or VHH-Fc, differing in one or more regions of their sequence. Antibody molecules typically have sequence variability in their complementarity determining regions (CDRs), which are the regions primarily involved in antigen recognition. A repertoire of bispecific binders as described herein may be a repertoire of bispecific antibody molecules which differ in one or more CDRs, for example there may be sequence diversity in all CDRs, or in one or more particular CDRs such as the heavy chain CDR3 and/or light chain CDR3.

For multimeric binders, e.g. dimeric bispecific binders, it is also encompassed to use repertoires of bispecific binders wherein one or more subunits do not have sequence variability. In other words, for example in the case of dimeric bispecific binders, one subunit may be kept constant while the other subunit may have one or more regions of amino acid sequence diversity as described above.

It is also encompassed to use repertoires of bispecific binders differing with respect to one or more linkers. “Linkers” are described elsewhere herein in more detail and include, for example, the linkers linking the VH and VL domains of scFvs and the linkers linking a binding domain to an Fc domain. Accordingly, for example, a repertoire of bispecific binders as described herein may be a repertoire of bispecific antibody molecules which have the same CDR or the same variable region, but which differ with respect to a linker sequence. Using repertoires of bispecific binders differing in one or more linkers allows the optimization of molecules in their final desired (therapeutic) format with respect to linker length and/or sequence.

It is also encompassed to use repertoires of bispecific binders comprising humanized and/or low immunogenicity variants of bispecific binders. Using such repertoires of humanized/low immunogenicity variants of bispecific binders allows the optimization of humanized/low immunogenic molecules in their final desired (therapeutic) format. Techniques for antibody humanization and reducing immunogenicity are known to the skilled person, as described for example in Safdari et al. Antibody humanization methods—a review and update. Biotechnol Genet Eng Rev. 2013; 29:175-86; and Ministro et al. Therapeutic Antibody Engineering and Selection Strategies. Adv Biochem Eng Biotechnol. 2020; 171:55-86, both incorporated herein by reference.

For multimeric binders, e.g. dimeric bispecific binders, donor DNA encoding the bispecific binder may be provided as one or more DNA molecules. For example, where individual bispecific antibody subunits are to be separately expressed, these may be encoded on separate molecules of donor DNA. The donor DNA integrates into the cellular DNA at multiple integration sites, e.g., the first subunit at one locus and the second subunit at a second locus. Methods of introducing donor DNA encoding separate bispecific binder subunits are described in more detail elsewhere herein. Alternatively, and preferably, both subunits of a multimeric, e.g. dimeric, bispecific binder may be encoded on the same molecule of donor DNA which integrates in the cellular DNA, optionally at a fixed locus.

A bispecific binder may be a bispecific antibody molecule or a bispecific non-antibody protein that comprises two or more antigen-binding sites. An antigen binding site may be provided by means of arrangement of peptide loops on non-antibody protein scaffolds such as fibronectin or cytochrome B etc., or by randomising or mutating amino acid residues of a loop within a protein scaffold to confer binding to a desired target (Haan & Maggos. BioCentury 2004; 12(5): A1-A6; Koide et al. Journal of Molecular Biology 1998; 284:1141-1151; Nygren et al. Current Opinion in Structural Biology 1997; 7:463-469). Protein scaffolds for antibody mimics are disclosed in WO0034784 in which proteins (antibody mimics) are described that include a fibronectin type III domain having at least one randomised loop. A suitable scaffold into which to graft one or more peptide loops, e.g., a set of antibody VH CDR loops, may be provided by any domain member of the immunoglobulin gene superfamily. The scaffold may be a human or non-human protein.

In addition to antibody sequences and/or an antigen-binding site, a bispecific binder may comprise other amino acids, e.g., forming a peptide or polypeptide, such as a folded domain, or to impart to the molecule another functional characteristic in addition to the ability to bind antigen. A bispecific binder may carry a detectable label, or may be conjugated to a toxin or a targeting moiety or enzyme (e.g., via a peptidyl bond or linker). For example, a bispecific binder may comprise a catalytic site (e.g., in an enzyme domain) as well as an antigen binding site, wherein the antigen binding site binds to the antigen and thus targets the catalytic site to the antigen. The catalytic site may inhibit biological function of the antigen, e.g., by cleavage.

Bispecific antibodies are preferred bispecific binders. Over 100 different bispecific antibody formats are known (Brinkmann & Kontermann. MAbs 2017; 9(2): 182-212). Bispecific antibodies, as used herein, have defined specificities and are artificial or recombinant molecules that are not found in nature. Preferably, a bispecific antibody as described herein is a dimeric bispecific antibody, i.e. consisting of two separate polypeptide chains or “subunits”.

As indicated elsewhere, preferred bispecific antibodies as described herein comprise a first binding domain coupled to a first Fc domain and a second binding domain coupled to a second Fc domain.

A binding domain may be coupled to an Fc domain either directly or indirectly, i.e., by using a “linker” or “linker peptide”. Particularly in the context of VHH binding domains (described in detail elsewhere herein), the binding domain may be coupled to the Fc domain through a linker. The skilled person can design appropriate linker lengths and linker sequences, for example depending on the target(s) that is (are) bound by the binding domain(s). In preferred embodiments, a linker coupling a binding domain to an Fc domain is a flexible linker. The person skilled in the art understands that flexible linkers are generally composed of small, non-polar (e.g., glycine) or polar (e.g., serine and threonine) amino acids, allowing them to provide flexibility and mobility of the connecting functional domains (as reviewed in Chen et al., Adv Drug Deliv Rev 2013; 65(10): 1357-1369; Chichili et al. Protein Sci. 2013; 22(2): 153-67, both incorporated herein by reference). For example, suitable flexible linkers include (GGGGS)n (SEQ ID NO: 25), wherein “n” is the number of repeats which may optionally be 2-7, preferably 3-6.

A bispecific antibody comprising one binding site for each antigen may be denoted as a bivalent antibody. For example, the preferred bispecific antibodies of this disclosure comprising a first binding domain coupled to a first Fc domain and a second binding domain coupled to a second Fc domain may be denoted as bivalent bispecific antibodies. Such bivalent bispecific antibodies may be described as having a “1+1” stoichiometry.

However, this disclosure also encompasses multivalent bispecific antibodies. For example, adding an additional binding domain to each of the polypeptide chains of a bivalent bispecific antibody as described herein results in tetravalent molecules with a “2+2” stoichiometry. Other formats allow to generate “1+2” or “1+3” molecules, having one binding site for one antigen and 2 or 3 binding sites for the other antigen, respectively.

Accordingly, in some embodiments, a bispecific antibody as described herein comprises:
1, 2, 3 or 4 binding domains coupled to a first Fc domain; and
1, 2, 3 or 4 binding domains coupled to a second Fc domain.

Bispecific antibodies as used herein may have either a symmetric or an asymmetric architecture, preferably an asymmetric architecture. Bispecific antibodies having a symmetric architecture may be understood to be such that the binding domains coupled to the first Fc domain and the binding domains coupled to the second Fc domain are of the same type, while bispecific antibodies having an asymmetric architecture may be understood to be such that the binding domains coupled to the first Fc domain and the binding domains coupled to the second Fc domain are of a different type. In the context of the preferred bispecific antibodies of this disclosure comprising a first binding domain coupled to a first Fc domain and a second binding domain coupled to a second Fc domain, bispecific antibodies having a symmetric architecture may be understood to have a first and second binding domain of the same type, while bispecific antibodies having an asymmetric architecture may be understood to have a first and second binding domain of a different type. Suitable types of binding domains in the context of this disclosure are described below.

### Binding Domain

A binding domain may be any antibody or antibody fragment or antibody domain or the like capable of binding an antigen. A binding domain, as used herein, may thus alternatively be referred to as an antigen-binding domain.

In conventional antibodies, both the light and heavy chains are divided into “constant” and “variable” regions based on their structural and functional homology. The variable domains of both the light (VL) and heavy (VH) chain portions determine antigen recognition and specificity. Conversely, the constant domains of the light chain (CL) and the heavy chain (CH1, CH2 or CH3) confer other biological properties such as secretion, transplacental mobility, Fc receptor binding, complement binding, and the like. By convention the numbering of the constant region domains increases as they become more distal from the antigen binding site or amino terminus of the antibody. The N-terminal portion is a variable region and at the C-terminal portion is a constant region; the CH3 and CL domains actually comprise the carboxy-terminus of the heavy and light chains, respectively.

In addition to conventional antibodies, made of two heavy and two light chains, certain vertebrates, notably Camelidae including dromedaries, camels, llamas, and alpacas; as well as some cartilaginous fishes such as sharks, produce so called heavy-chain antibodies that are antibodies made only of two heavy chains and lacking the two light chains present in conventional antibodies. In Camelidae, these are designated as VHHs for variable domain of heavy-chain antibodies and in some cartilaginous fishes such as sharks, these are designated as VNARs for variable new antigen receptor.

Preferred binding domains in this disclosure are single-chain binding domains, i.e. the binding domains are monomeric.

In some embodiments, a binding domain comprises at least one antibody variable domain. In some embodiments, the first and/or second binding domain comprises at least one antibody variable domain.

In some embodiments, a binding domain comprises a single antibody variable domain. In some embodiments, the first and/or second binding domain comprises a single antibody variable domain. Binding domains comprising a single antibody variable domain may also be denoted as single domain antibodies (sdAb). An sdAb is a small monomeric antigen-binding fragment of an antibody, namely the variable region of an antibody heavy or light chain.

Heavy-chain antibodies (HCAb) found in Camelidae are composed of two heavy chains divided in three domains each: CH3-CH2-VHH. The variable domain of HCAb corresponding to the paratope recognizing the antigen is called VHH. This variable domain can be expressed on its own and still recognize the antigen. The amino acid sequence and structure of a VHH can be considered, without however being limited thereto, to be comprised of four framework regions or ‘FR's’, which are referred to in the art and herein below as ‘framework region 1’ or ‘FR1’; as ‘framework region 2’ or ‘FR2’; as ‘framework region 3’ or ‘FR3’; and as ‘framework region 4’ or ‘FR4’, respectively, which framework regions are interrupted by three complementary determining regions or ‘CDR's’, which are referred to in the art as ‘complementarity determining region 1’ or ‘CDR1’; as ‘complementarity determining region 2’ or ‘CDR2’; and as ‘complementarity determining region 3’ or ‘CDR3’, respectively.

Heavy-chain antibodies (HCAb) found in sharks are composed of two heavy chains made of five constant domains (CNAR1, CNAR2, CNAR3, CNAR4 and CNAR5). VNARs are the variable domain of these antibodies. As for VHHs, VNARs bear full antigen recognition properties. The main difference of their variable domain is the absence of complementary-determining region 2 (CDR2) leading to only two CDRs.

In some embodiments, a binding domain comprising a single antibody variable domain may be (i) a variable domain of the heavy chain of a heavy chain antibody, which is naturally devoid of light chains, including but not limited to the variable domain of the heavy chain of heavy-chain antibodies of camelids (VHH) or sharks (VNAR) or (ii) the variable domain of the heavy chain of a conventional four-chain antibody, including but not limited to a camelized variable domain of the heavy chain of a conventional four-chain.

In preferred embodiments, a binding domain comprising a single antibody variable domain is a VHH or a VNAR, preferably a VHH.

In some embodiments, a binding domain comprises two antibody variable domains. In some embodiments, the first and/or second binding domain comprises two antibody variable domains. Binding domains comprising two single antibody variable domains are preferably single-chain variable fragments (scFv).

scFv molecules consist of a VH domain and a VL domain joined by a linker or linker peptide. In preferred embodiments, a linker coupling a VH domain and a VL domain is a flexible linker. Suitable flexible linkers are described herein elsewhere. In the scFv molecule, the VH and VL domains form a VH-VL pair in which the complementarity determining regions of the VH and VL come together to form an antigen binding site.

In some embodiments, the 1, 2, 3 or 4 binding domains coupled to a first Fc domain comprise a single antibody variable domain as described herein. In some embodiments, the 1, 2, 3 or 4 binding domains coupled to a second Fc domain comprises two antibody variable domains as described herein. In some embodiments, the 1, 2, 3 or 4 binding domains coupled to a first Fc domain comprise a single antibody variable domain as described herein, and the 1, 2, 3 or 4 binding domains coupled to a second Fc domain comprise two antibody variable domains as described herein, or vice versa.

In some embodiments, the first binding domain comprises a single antibody variable domain as described herein.

In some embodiments, the second binding domain comprises two antibody variable domains as described herein.

As explained above, it is preferred that the first and second binding domain are of a different type. Accordingly, in preferred embodiments, the first binding domain comprises a single antibody variable domain as described herein (e.g. a VHH), and the second binding domain comprises two antibody variable domains as described herein (e.g. an scFv), or vice versa.

### Constant Domain

As indicated elsewhere in this document, bispecific antibodies described herein may comprise:
1, 2, 3 or 4 binding domains coupled to a first Fc domain; and
1, 2, 3 or 4 binding domains coupled to a second Fc domain.

For example, preferred bispecific antibodies as described herein comprise a first binding domain coupled to a first Fc domain and a second binding domain coupled to a second Fc domain. The terms Fc region, Fc domain and the like may be used interchangeably herein, and refer to the CH2 and CH3 domains of a canonical IgG antibody molecule. The CH2 and CH3 domains are responsible for the interactions with effector cells and complement components within the immune system. Thus, bispecific antibodies comprising an Fc domain have the advantage that they may display Fc-mediated effector functions, such as antibody-dependent cell-mediated cytotoxicity (ADCC), antibody-dependent cellular phagocytosis (ADCP), complement fixation, and FcRn-mediated recycling.

In preferred embodiments, the Fc domains are engineered to promote heterodimerization. Typically, this means that the first Fc domain and the second Fc domain are different (i.e., they have a different amino acid sequence). Thus, in some embodiments, the first and second Fc domain as described herein are different from each other. In some embodiments, the first and second Fc domain as described herein are different from each other and are engineered to promote heterodimerization. Various strategies for promoting heterodimerization are known to the skilled person, based on either steric or electrostatic steering effects, or a combination thereof, as well as formation of defined interchain disulfides, to generate a complementary interface favoring heterodimerization over homodimerization. Several approaches have been described and may be suitable in the context of this disclosure, see e.g. Table 1 in Brinkmann & Kontermann. MAbs 2017; 9(2): 182-212:

| | CH3 | CH3 | disulfide | |
|---|---|---|---|---|
| strategy | domain 1 | domain 2 | bond in CH3 | References |
| knobs-into-holes | T366Y | Y407T | no | Ridgway J B, Presta L G, Carter |
| (Y-T) | | | | P. ‘Knobs-into-holes' engineering of |
| | | | | antibody CH3 domains for heavy |
| | | | | chain heterodimerization. Protein Eng |
| | | | | 1996; 9: 617-21; PMID: 8844834 |
| knobs-into-holes | S354C, | Y349C, | yes | Atwell S, Ridgway J B, Wells J A, |
| (CW-CSAV) | T366W | T366S, | | Carter P. Stable heterodimers from |
| | | L368A, | | remodeling the domain interface of a |
| | | Y407V | | homodimer using a phage display |
| | | | | library. J Mol Biol 1997; 270: 26-35 |
| | | | | Merchant A M, Zhu Z, Yuan J Q, |
| | | | | Goddard A, Adams C W, Presta L G, |
| | | | | Carter P. An efficient route to human |
| | | | | bispecific IgG. Nat Biotechnol |
| | | | | 1998; 16: 677-681; |
| HA-TF | S364H, | Y349T, | | Moore G L, Bautista C, Pong E, |
| | F405A | T394F | | Nguyen D H, Jacinto J, Eivaci A, |
| | | | | Muchhal U S, Karki S, Chu S Y, Lazar |
| | | | | G A. A novel bispecific antibody format |
| | | | | enables simultaneous bivalent and |
| | | | | monovalent co-engagement of distinct |
| | | | | target antigens. MAbs 2011; 3: 546-557 |
| ZW1 | T350V, | T350V, | no | Von Kreudenstein T S, Escobar- |
| (VYAV-VLLW) | L351Y, | T366L, | | Carbrera E, Iario P I, D'Angelo I, Brault |
| | F405A, | K392L, | | K, Kelly J, Durocher Y, Baardsnes J, |
| | Y407V | T394W | | Woods R J, Xie M H, et al.. Improving |
| | | | | biophysical properties of a bispecific |
| | | | | antibody scaffold to aid developability: |
| | | | | quality by molecular design. |
| | | | | MAbs 2013; 5: 646-54 |
| CH3 charge | K392D, | E356K, | no | Gunasekaran K, Pentony M, Shen M, |
| pairs | K409D | D399K | | Garrett L, Forte C, Woodward A, Ng |
| (DD-KK) | | | | S B, Born T, Retter M, Manchulenko K, |
| | | | | et al.. Enhancing antibody Fc |
| | | | | heterodimer formation through |
| | | | | electrostatic steering effects: |
| | | | | applications to bispecific molecules |
| | | | | and monovalent IgG. J Biol Chem |
| | | | | 2010; 285: 19637-46 |
| IgG1 | IgG1: | IgG1: | no | Strop P, Ho W H, Boustany L M, |
| hinge/CH3 | D221E, | D221R, | | Abdiche Y N, Lindquist K C, Farias S E, |
| charge | P228E, | P228R, | | Rickert M, Appah C T, Pascua E, |
| pairs | L368E | K409R | | Radcliffe T, et al.. Generating |
| (EEE-RRR) | | | | bispecific human IgG1 and IgG2 |
| | | | | antibodies from any antibody pair. |
| | | | | J Mol Biol 2012; 420: 204-19 |
| IgG2 | IgG2: | IgG2: | no | Strop P, Ho W H, Boustany L M, |
| hinge/CH3 | C223E, | C223R, | | Abdiche Y N, Lindquist K C, Farias S E, |
| charge | P228E, | E225R, | | Rickert M, Appah C T, Pascua E, |
| pairs | L368E | P228R, | | Radcliffe T, et al.. Generating |
| (EEE-RRRR) | | K409R | | bispecific human IgG1 and IgG2 |
| | | | | antibodies from any antibody pair. |
| | | | | J Mol Biol 2012; 420: 204-19 |
| EW-RVT | K360E, | Q347R, | no | Choi H J, Kim Y J, Lee S, Kim Y S. A |
| | K409W, | D399V, | | heterodimeric Fc-based bispecific |
| | | F405T | | antibody simultaneously targeting |
| | | | | VEGFR-2 and Met exhibits potent |
| | | | | antitumor activity. Mol Cancer Ther |
| | | | | 2013; 12: 2748-59 |
| EW-RVT_{S-S} | K360E, | Q347R, | yes | Choi H J, Seok S H, Kim Y J, Seo M d, |
| | K409W, | D399V, | | Kim Y S. Crystal structures of |
| | Y349C | F405T, | | immunoglobulin Fc heterodimers |
| | | S354C | | reveal the molecular basis for |
| | | | | heterodimer formation. |
| | | | | Mol Immunol 2015; 65: 377-83 |
| Biclonic | 366K | 351D or | no | Geuijen C, Rovers E, Nijhuis R, Visser |
| | (+351K) | E or D at | | T, den Blanken-Smit R, Barelink W, |
| | | 349, | | et al.. Preclinical activity of MCLA- |
| | | 368, | | 128, an ADCC enhanced human |
| | | 349, or | | bispecific IgG1 antibody targeting the |
| | | 349 + | | HER2:HER3 heterodimer. Journal of |
| | | 355 | | Clinical Oncology 2014; 32: suppl: 560 |
| DuoBody | F405L | K409R | no | Labrijn A F, Meesters J I, de Goeij B E, |
| (L-R) | | | | van den Bremer E T, Neijssen J, van |
| | | | | Kampen M D, Strumane K, Verploegen |
| | | | | S, Kundu A, Gramer M J, et al.. |
| | | | | Efficient generation of stable |
| | | | | bispecific IgG1 by controlled Fab-arm |
| | | | | exchange. Proc Natl Acad Sci USA |
| | | | | 2013; 110: 5145-50 |
| SEEDbody | IgG/A | IgG/A | no | Davis J H, Aperlo C, Kurosawa E, Lan |
| | chimera | chimera | | Y, Lo K M, Huston J S. SEEDbodies: |
| | | | | fusion proteins based on strand- |
| | | | | exchange engineered domain (SEED) |
| | | | | CH3 heterodimers in an Fc analogue |
| | | | | platform for asymmetric binders or |
| | | | | immunofusions and bispecific |
| | | | | antibodies. Protein Eng Des Sel |
| | | | | 2010; 23: 195-202 |
| BEAT | residues | residues | no | Moretti P, Skegro D, Ollier R, |
| | from | from | | Wassmann P, Aebischer C, Laurent |
| | TCRα | TCRβ | | T, et al.. BEAT the bispecific |
| | interface | interface | | challenge: a novel and efficient |
| | | | | platform for the expression of |
| | | | | bispecific IgGs. BMC Proceedings |
| | | | | 2013; 7(Suppl 6): O9 |
| 7.8.60 | K360D, | E345R, | no | Leaver-Fey A, Froning K J, Atwell S, |
| (DMA-RRVV) | D399M, | Q347R, | | Aldaz H, Pustilnik A, Lu F, Huang F, |
| | Y407A | T366V, | | Yuan R, Hassanali S, Chamberlain |
| | | K409V | | A K, et al.. Computationally Designed |
| | | | | Bispecific Antibodies using Negative |
| | | | | State Repertoires. Structure 2016; |
| | | | | 24: 641-51 |
| 20.8.34 | Y349S, | E356G, | no | Leaver-Fey A, Froning K J, Atwell S, |
| (SYMV-GDQA) | K370Y, | E357D, | | Aldaz H, Pustilnik A, Lu F, Huang F, |
| | T366M, | S364Q, | | Yuan R, Hassanali S, Chamberlain |
| | K409V | Y407A | | A K, et al.. Computationally Designed |
| | | | | Bispecific Antibodies using Negative |
| | | | | State Repertoires. Structure 2016; |
| | | | | 24: 641-51 |

In some embodiments, the CH3 domain of the first Fc domain and the CH3 domain of the second Fc domain have corresponding mutations that promote dimerization. Such mutations may for example be selected from the table below.

| CH3 domain 1 | CH3 domain 2 |
|---|---|
| T366Y | Y407T |
| T366W | T366S, L368A, Y407V |
| S354C, T366W | Y349C, T366S, L368A, Y407V |
| S364H, F405A | Y349T, T394F |
| T350V, L351Y, F405A, Y407V | T350V, T366L, K392L, T394W |
| K392D, K409D | E356K, D399K |
| K360E, K409W | Q347R, D399V, F405T |
| K360E, K409W, Y349C | Q347R, D399V, F405T, S354C |
| 366K (+351K) | 351D or E or D at 349, 368, 349, |
| | or 349 + 355 |
| F405L | K409R |
| residues from TCRα interface | residues from TCRβ interface |
| K360D, D399M, Y407A | E345R, Q347R, T366V, K409V |
| Y349S, K370Y, T366M, K409V | E356G, E357D, S364Q, Y407A |

The knob-into-hole technique is based on creating a knob at the CH3 domain interface of the first chain by replacing one or more smaller amino acid side chains with larger ones (for example, T366Y or T366W or S354C+T366W); and creating a hole in the juxtaposed position at the CH3 interface of the second chain by replacing one or more larger amino acid side chains with smaller ones (for example, Y407T or T366S+L368A+Y407V or Y349C+T366S+L368A+Y407V). Because knob-knob and hole-hole interactions are sterically hindered or energetically less favorable, heterodimer formation is favored.

In preferred embodiments, the first and second Fc domain, in particular the CH3 region of the first and second Fc domains, comprises corresponding knob and hole mutations. Thus, the first Fc domain may comprise a knob mutation and the second Fc domain may comprise a hole mutation, or the first Fc domain may comprise a hole mutation and the second Fc domain may comprise a knob mutation.

A knob mutation may be selected from the group consisting of: T366Y, T366W, and S354C+T366W. A hole mutation may be selected from the group consisting of: Y407T, T366S+L368A+Y407V, and Y349C+T366S+L368A+Y407V. Corresponding knob and hole mutations may be selected from the group consisting of:
T366Y (knob) and Y407T (hole);
T366W (knob) and T366S+L368A+Y407V (hole); and
S354C+T366W (knob) and Y349C+T366S+L368A+Y407V (hole).

In preferred embodiments, an Fc domain comprising a knob mutation is coupled with a binding domain comprising a single antibody variable domain as described herein (e.g. a VHH) and/or an Fc domain comprising a hole mutation is coupled with a binding domain comprising two antibody variable domains as described herein (e.g. an scFv). In more preferred embodiments, an Fc domain comprising a knob mutation is coupled with a VHH) and an Fc domain comprising a hole mutation is coupled with an scFv.

Bispecific binders may be (derived from) human antibody molecules. Thus, where constant domains are present these are preferably human constant domains.

A library as described herein may be used to select a bispecific antibody molecule that binds one or more antigens of interest. Selection from libraries is described in detail below. The selected bispecific antibody molecules as well as their encoding nucleic acids and the sequences thereof are an aspect of the present disclosure. Antibody molecules and their encoding nucleic acid may be provided in isolated form, or as cell clones containing said nucleic acid and expressing said molecules.

Bispecific antibody molecules may be selected from a library and then modified, for example the in vivo half-life of an antibody molecule can be increased by chemical modification, for example PEGylation, or by incorporation in a liposome.

### Sources of Nucleic Acids Encoding Binder Domains

VH and VL genes could be amplified from the B cells of immunised animals and cloned into an appropriate vector for introduction into eukaryotic libraries as described herein. Phage display and ribosome display allows very large libraries (>10\hat{ }9 clones) to be constructed enabling isolation of human antibodies without immunisation. Producing libraries according to the present disclosure could also be used in conjunction with such methods. Following rounds of phage display selection, the selected population of binders could be introduced into eukaryotic cells by nuclease-directed integration as described herein. This would allow the initial use of very large libraries based in other systems (e.g., phage display) to enrich a population of binders while allowing their efficient screening using eukaryotic cells as described above. Thus the disclosure can combine the best features of both phage display and eukaryotic display to give a high throughput system with quantitative screening and sorting.

Using phage display and yeast display it has previously been demonstrated that it is also possible to generate binders without resorting to immunisation, provided display libraries of sufficient size are used. For example multiple binders were generated from a non-immune antibody library of >10\hat{ }7 clones (Marks et al. J Mol Biol 1991; 222(3), 581-597). This in turn allows generation of binders to targets which are difficult by traditional immunisation routes e.g., generation of antibodies to “self-antigens” or epitopes which are conserved between species. For example, human/mouse cross-reactive binders can be enriched by sequential selection on human and then mouse versions of the same target. Since it is not possible to specifically immunise humans to most targets of interest, this facility is particularly important in allowing the generation of human antibodies which are preferred for therapeutic approaches.

In examples of mammalian display to date, where library sizes and quality were limited, binders have only been generated using repertoires which were pre-enriched for binders, e.g., from immunisation or from engineering of pre-existing binders. The ability to make large libraries in eukaryotic cells and particularly higher eukaryotes creates the possibility of isolating binders direct from these libraries starting with non-immune binders or binders which have not previously been selected within another system. By producing a library according to the present disclosure it is possible to generate binders from non-immune sources. This in turn opens up the possibilities for using binder genes from multiple sources. Binder genes could come from PCR of natural sources such as antibody genes. Binder genes could also be re-cloned from existing libraries, such as antibody phage display libraries, and cloned into a suitable donor vector for nuclease-directed integration into target cells. Binders may be completely or partially synthetic in origin. Furthermore various types of binders are described elsewhere herein, for example binder genes could encode antibodies or could encode alternative scaffolds (Skerra. Curr Opin Biotechnol 2007; 18(4): 295-304; Gebauer & Skerra. Curr Opin Chem Biol 2009; 13(3): 245-255), peptides or engineered proteins or protein domains.

### Eukaryotic Cells

Preferred eukaryotic cells and eukaryotic cell clones for aspects of this disclosure including the methods, uses and libraries as described herein are defined below. It is understood that all preferences relating to eukaryotic cells may also be applied to eukaryotic cell clones.

Eukaryotic cells are preferably higher eukaryotic cells, defined here as cells with a genome greater than that of Saccharomyces cerevisiae which has a genome size of 12×10\hat{ }6 base pairs (bp). The higher eukaryotic cells may for example have a genome size of greater than 2×10\hat{ }7 base pairs. In some embodiments, the eukaryotic cells are higher eukaryotic cells with a genome size of greater than 2×10\hat{ }7 base pairs. This includes, for example, mammalian, avian, insect or plant cells, preferably mammalian cells. Preferably eukaryotic cells are mammalian cells, e.g., mouse or human. More preferably, eukaryotic cells are human cells. The eukaryotic cells may be primary cells or may be cell lines. Chinese hamster ovary (CHO) cells are commonly used for antibody and protein expression but any alternative stable cell line such as HEK293 cells may be used in the disclosure. Methods are available for efficient introduction of foreign DNA into primary cells allowing these to be used (e.g., by electroporation where efficiencies and viabilities up to 95% have been achieved, Parthiban et al. MAbs 2019; 11(5); 884-898; Dyson et al. MAbs 2020; 12(1): 1829335).

A particular benefit of nuclease-directed integration comprised in a method for generating a library relates to integration of binder genes into higher eukaryotic cells with larger genomes where homologous recombination in the absence of nuclease cleavage is less effective. Yeast (e.g., Saccharomyces cerevisiae) has a smaller genome than mammalian cells and homologous recombination directed by homology arms (in the absence of nuclease-directed cleavage) is an effective way of introducing foreign DNA compared to higher eukaryotes. Nuclease-directed integration has been used in yeast cells to solve the problem of efficient integration of multiple genes into individual yeast cells, e.g., for engineering of metabolic pathways (US2012/0277120), but this work does not incorporate introduction of libraries of binders nor does it address the problems of library construction in higher eukaryotes.

Preferred eukaryotic cells are T lymphocyte lineage cells (e.g., primary T cells or a T cell line) or B lymphocyte lineage cells. Of particular preference are primary T-cells or T cell derived cell lines for use in TCR libraries including cell lines which lack TCR expression (Letourneur, F., Malissen, B. European Journal of Immunology 1989; 19(12): 2269-2274; Kanayama et al. Biochem Biophys Res Commun 2005; 327(1), 70-75; Lin et al. Nucleic Acids Research 2011; 39(3), e14. Preferred B lymphocyte lineage cells are B cells, pre-B cells or pro-B cells and cell lines derived from any of these.

Construction of libraries in primary B cells or B cell lines would be of particular value for construction of antibody libraries. These eukaryotic cells are preferred in methods for producing a library. Breous-Nystrom et al. Methods 2014; 65(1): 57-67 have generated libraries in a murine pre-B cell line (1624-5). The chicken B cell derived cell line DT40 (ATCC CRL-2111) has particular promise for construction of libraries of binders. DT40 is a small cell line with a relatively rapid rate of cell division. Repertoires of binders could be targeted to specific loci using zinc finger nucleases (ZFNs), TALE nucleases or CRISPR/Cas9 targeted to endogenous sequences or by targeting pre-integrated heterologous sites which could include meganuclease recognition sites. DT40 cells express antibodies and so it will be advantageous to target antibody genes within the antibody locus either with or without disruption of the endogenous chicken antibody variable domains. DT40 cells have also been used as the basis of an in vitro system for generation of chicken IgMs termed the Autonomously Diversifying Library system (ADLib system) which takes advantage of intrinsic diversification occurring at the chicken antibody locus. As a result of this endogenous diversification it is possible to generate novel specificities. The nuclease-directed approach described here could be used in combination with ADLib to combine diverse libraries of binders from heterologous sources (e.g., human antibody variable region repertoires or synthetically derived alternative scaffolds) with the potential for further diversification with the chicken IgG locus. Similar benefits could apply to human B cell lines such as Nalm6 (Adachi et al. DNA and Cell Biology 2006; 25(1), 19-24).

Other preferred B lineage cell lines preferred in methods for identifying a locus and for producing a library include lines such as the murine pre-B cell line 1624-5 and the pro-B cell line Ba/F3. Ba/F3 is dependent on IL-3 (Palacios et al. Cell 1985; 41(3), 727-734) and its use is discussed elsewhere herein.

Finally a number of human cell lines are preferred including those listed in the “Cancer Cell Line Encyclopaedia” (Barretina et al. Nature 2012; 483 (7391): 603-607) or “COSMIC catalogue of somatic mutations in cancer” (Forbes et al. Nucleic Acids Research 2011; 39 (Database issue): D945-50).

In a method for producing a library as well as in libraries as described herein, the eukaryotic cells are preferably of a single type of cells, produced by introduction of donor DNA into a population of clonal eukaryotic cells, for example by introduction of donor DNA into cells of a particular cell line. The main significant difference between the different library clones will then be due to integration of the donor DNA.

### Eukaryotic Viral Systems

The advantages of the aspects and embodiments described herein, such as the methods for producing a library of eukaryotic cell clones, and the resulting libraries, could be applied to viral display systems based around eukaryotic expression systems, e.g., baculoviral display or retroviral display (Russell et al. Nucleic Acids Res 1993; 21(5): 1081-1085; Boublik et al. Nature Biotech 1995; 13(10): 1079-1084; Mottershead et al. Biochemical and Biophysical Research Communications 2000; 275(&): 84-90; Oker-Blom et al. Briefings in functional genomics and proteomics 2003; 2(3): 244-253). In this approach each cell will encode a binder capable of being incorporated into a viral particle. In the case of retroviral systems the encoding mRNA would be packaged and the encoded binder would be presented on the cell surface. In the case of baculoviral systems, genes encoding the binder would need to be encapsulated into the baculoviral particle to maintain an association between the gene and the encoded protein. This could be achieved using host cells carrying episomal copies of the baculoviral genome. Alternatively integrated copies could be liberated following the action of a specific nuclease (distinct from the one used to drive site-specific integration). In the case of multimeric binder molecules some partners could be encoded within the cellular DNA with the genes for one or more partners being packaged within the virus.

### Introduction of Nucleic Acids

Methods described herein comprise the introduction of nucleic acids into a eukaryotic cell. In a method for generating a library, donor DNA molecules are introduced. Unless specifically mentioned otherwise, the introduction of a nucleic acid refers to the introduction of a DNA molecule in a eukaryotic cell.

Numerous methods have been described for introducing nucleic acids into eukaryotic cells, including transfection, infection or electroporation. These methods are well-known to the person of skill in the art, see for example Green & Sambrook, Molecular Cloning: A Laboratory Manual, 4th edition, Cold Spring Harbor Press, New York (2012) (ISBN 978-1-936113-42-2); and Ausubel et al., Current Protocols in Molecular Biology, 3rd edition, John Wiley & Sons Inc (2003).

Transfection of large numbers of cells is possible by standard methods including polyethyleneimine-mediated transfection as described herein. In addition methods are available for highly efficient electroporation of 10¹⁰ cells in 5 minutes, see e.g. Parthiban et al. MAbs 2019; 11(5); 884-898; Dyson et al. MAbs 2020; 12(1): 1829335.

In a method for generating a library, combinatorial libraries could be created wherein members of multimeric binding pairs (e.g., a first and a second subunit as described herein) or even different parts of the same binder molecule are introduced on different plasmids. Introduction of separate donor DNA molecules encoding separate binders or binder subunits may be done simultaneously or sequentially. For example a first subunit of a multimeric bispecific binder could be introduced by transfection or infection, the cells grown up and selected if necessary. Other components, such as a second subunit of a multimeric bispecific binder could then be introduced in a subsequent infection or transfection step. One or both steps could involve nuclease-directed integration to specific genomic loci.

### Integration of Nucleic Acids

A method for generating a library as described herein may involve the integration of nucleic acids into the genome of the eukaryotic cell. In this context, the terms genome and cellular DNA may be used interchangeably. Unless explicitly mentioned otherwise, integration refers to the integration of a DNA molecule into the genome of a eukaryotic cell. The nucleic acid is integrated into the genome (i.e. cellular DNA), forming recombinant DNA having a contiguous DNA sequence in which the nucleic acid is inserted at the integration site. In the present disclosure, integration is mediated by the natural DNA repair mechanisms that are endogenous to the cell.

Integration of a nucleic acid may be random or specific.

In random integration of a nucleic acid, the integration site is not defined by a specific sequence.

In specific integration of a nucleic acid the integration site is defined by a specific sequence. The nucleic acid in the context of specific integration may be called the donor DNA, the donor DNA molecule or the donor DNA sequence or the like.

Specific integration can be allowed to occur by introducing the nucleic acid into a cell, allowing the site-specific nuclease to create an integration site, and allowing the donor DNA to be integrated. In this context, specific integration may also be called nuclease-directed integration. Cells may be kept in culture for sufficient time for the DNA to be integrated. This will usually result in a mixed population of cells, including (i) recombinant cells into which the donor DNA has integrated at the integration site created by the site-specific nuclease, and optionally (ii) cells in which donor DNA has integrated at sites other than the desired integration site and/or optionally (iii) cells into which donor DNA has not integrated. The desired recombinant cells and the resulting clones may thus be provided in a mixed population further comprising other eukaryotic cells. Selection methods described elsewhere herein may be used to select the desired cells and clones, or to enrich said mixed population in said desired cells and clones. As explained above, integration is mediated by the natural DNA repair mechanisms that are endogenous to the cell. Endogenous DNA repair mechanisms in eukaryotic cells include homologous recombination, non-homologous end joining (NHEJ) and microhomology-mediated end joining (MMEJ). The efficiency of integration by such processes can be increased by the introduction of double stranded breaks (DSBs) in the cellular DNA and efficiency gains of 40,000 fold have been reported using rare cutting endonucleases (meganucleases) such as I-Scel (Porteus & Baltimore. Science 2003; 300(5620):763; Rouet et al. Molecular and Cellular Biology 1994; 14(12):8096-8106; Jasin. Trends in genetics 1996; 12(6):224-228).

Unlike the site-specific recombination involved in systems such as the Flp-In system [16], integration in the present disclosure does not require exogenous recombinases or engineered recombinase recognition sites. Therefore, a method for generating a library preferably do not include a step of recombinase-mediated integration of a DNA molecule. Furthermore, the eukaryotic cells in a method for identifying a locus and in a method for generating a library preferably lack a recombination site for a site-specific recombinase. The mechanisms and practicalities of specific integration of donor DNA into cellular DNA by recombinases and nucleases are very distinct as discussed by Jasin 1996 (Jasin. Trends in genetics 1996; 12(6):224-228).

In contrast specific integration comprising the use of site-specific nuclease involves the nuclease act to create breaks or nicks within the cellular DNA, which are exposed to and repaired by endogenous cellular repair mechanisms such as homologous recombination or NHEJ. Recombinase-based approaches have an absolute requirement for pre-integration of their recognition sites, so such methods require engineering of the “hot spot” integration site into the cellular DNA as a preliminary step. With nuclease-directed integration it is possible to engineer nucleases or direct via guide RNA in the case of CRISPR: Cas9 to recognise endogenous recognition sequences, i.e., nucleic acid sequences occurring naturally in the cellular DNA. Finally, at a practical level nuclease-directed approaches are more efficient for specific integration of transgenes at the levels required to make large libraries of binders.

The DNA repair mechanism by which the donor DNA is integrated in a method as described herein such as a method for generating a library can be pre-determined or biased to some extent by design of the donor DNA and/or choice of site-specific nuclease.

Homologous recombination is a natural mechanism used by cells to repair double stranded breaks using homologous sequence (e.g., from another allele) as a template for repair. Homologous recombination has been utilised in cellular engineering to introduce insertions (including transgenes), deletions and point mutations into the genome. Homologous recombination is promoted by providing homology arms on the donor DNA. Hence, the donor DNA preferably comprises homology arms. The original approach to engineering higher eukaryotic cells typically used homology arms of 5-10 kb within a donor plasmid to increase efficiency of targeted integration into the site of interest. Homologous recombination is particularly suitable for eukaryotes such as yeast, which has a genome size of only 12.5×10\hat{ }6 bp, where it is more effective compared with higher eukaryotes with larger genomes e.g., mammalian cells with 3000×10\hat{ }6 bp.

Homologous recombination can also be directed through (Fujioka et al. Nucleic Acids Res 1993; 21(3): 407-412) nicks in cellular DNA and this could also serve as a route for nuclease-directed integration into cellular DNA. Hence, the integration of donor DNA comprised in a method as described herein such as a method for generating a library preferably comprises the introduction of nicks in the cellular DNA. Two distinct pathways have been shown to promote homologous recombination at nicked DNA. One is essentially similar to repair at double strand breaks, utilizing Rad51/Brca2, while the other is inhibited by Rad51/Brca2 and preferentially uses single-stranded DNA or nicked double stranded donor DNA (Davus & Maizels. PLos ONE 2011; 6(9): e23981). Non-homologous end-joining (NHEJ) is an alternative mechanism to repair double stranded breaks in the genome where the ends of DNA are directly re-ligated without the need for a homologous template. Nuclease-directed cleavage of genomic DNA can also enhance transgene integration via non-homology based mechanisms. NHEJ provides a simple means of integrating in-frame exons into intron or allows integration of promoter: gene cassettes into the genome. Use of non-homologous methods allows the use of donor vectors which lack homology arms thereby simplifying the construction of donor DNA.

It has been pointed out that short regions of terminal homology are used to re-join DNA ends and it was hypothesized that 4 bp of microhomology might be utilized for directing repairing at double strand breaks, referred to as microhomology-mediated end joining (MMEJ) (Jasin. Trends in genetics 1996; 12(6):224-228).

### Site-Specific Nuclease

In some embodiments, the aspects and embodiments described herein involve the use of site-specific nucleases and their recognition sequences. For example, a method for generating a library may involve providing a site-specific nuclease which cleaves a recognition sequence in cellular DNA. Preferred site-specific nucleases are defined below. It is understood that all preferences relating to site-specific nuclease may also be applied mutatis mutandis to the corresponding recognition sites.

The site-specific nuclease cleaves cellular DNA following specific binding to a recognition sequence, thereby creating an integration site for donor DNA. In this context, the terms site, target site, recognition site and recognition sequence may be used interchangeably. The nuclease may create a double strand break or a single strand break (a nick). Nuclease-mediated DNA cleavage enhances site-specific integration of binder genes through endogenous cellular DNA repair mechanisms.

In a method as described herein such as a method for generating a library, the eukaryotic cells used may contain endogenous sequences recognized by the site-specific nuclease or the recognition sequence may be engineered into the cellular DNA. Furthermore, the site-specific nuclease may be exogenous to the cells, i.e. not occurring naturally in cells of the chosen type.

In a method as described herein such as a method for generating a library, the site-specific nuclease can be introduced before, after or simultaneously with introduction of the donor DNA. It may be convenient for the donor DNA to encode the nuclease in addition to a binder, or on separate nucleic acid which is co-transfected or otherwise introduced at the same time as the donor DNA. Clones of a library may optionally retain nucleic acid encoding the site-specific nuclease, or such nucleic acid may be only transiently transfected into the cells.

Any suitable site-specific nuclease may be used within this disclosure. It may be a naturally occurring enzyme or an engineered variant. There are a number of known nucleases that are especially suitable, such as those which recognise, or can be engineered to recognise, sequences that occur only rarely in cellular DNA.

Preferably, the site-specific nuclease recognizes only one or two distinct recognition sequences. This is advantageous since this should ensure that only one or two molecules of donor DNA are integrated per cell.

Rarity of the sequence recognised by the site-specific nuclease is more likely if the recognition sequence is relatively long. Preferably, the recognition sequence has a length of at least 10, 11, 12, 13, 14, 15, 16, 17, 18, 19, 20, 21, 22, 23, 24, 25, 26, 27, 28, 29, or 30 nucleotides. Preferably, the recognition sequence has a length from 10 up to 40, 39, 38, 37, 36, 35, 34, 33, 32, 31, 30, 29, 28, 27, 26, 25, 24, 23, 22, 21 or 20 nucleotides, or from 12 up to 40, 39, 38, 37, 36, 35, 34, 33, 32, 31, 30, 29, 28, 27, 26, 25, 24, 23, 22, 21 or 20 nucleotides.

Preferred site-specific nucleases are meganucleases, zinc finger nucleases (ZFNs), TALE nucleases, and nucleic acid-guided (e.g., RNA-guided) nucleases such as the CRISPR/Cas system. Each of these produces double strand breaks although engineered forms are known which generate single strand breaks.

Meganucleases (also known as homing endonucleases) are nucleases which occur across all the kingdoms of life and recognise relatively long sequences (12-40 bp). Given the long recognition sequence they are either absent or occur relatively infrequently in eukaryotic genomes. Meganucleases are grouped into 5 families based on sequence/structure. (LAGLIDADG (SEQ ID NO: 2) family, GIY-YIG family, HNH family, His-Cys box family and PD-(D/E) XK family). The best studied family is the LAGLIDADG (SEQ ID NO: 2) family which includes the well characterised I-Scel meganuclease from Saccharomyces cerevisiae. I-Scel recognises and cleaves an 18 bp recognition sequence (5′ TAGGGATAACAGGGTAAT, SEQ ID NO: 1) leaving a 4 bp 3′ overhang. Another commonly used example is I-Crel which originates from the chloroplast of the unicellular green algae of Chlamydomonas reinhardtii, and recognizes a 22 bp sequence (Silva et al. Current Gene Therapy 2011; 11(1): 11-27). A number of engineered variants have been created with altered recognition sequences (Epinat et al. Topics in Current Genetics 2013; 23:147-185). Meganucleases represent the first example of the use of site-specific nucleases in genome engineering [Rouet et al. Molecular and Cellular Biology 1994; 14(12): 8096-8106; Jasin. Trends in genetics 1996; 12(6): 224-228]. As with recombinase-based approaches, use of I-Scel and other meganucleases requires prior insertion of an appropriate recognition sequence to be targeted within the genome or engineering of meganucleases to recognize endogenous recognition sequences (Silva et al. Current Gene Therapy 2011; 11(1): 11-27). By this approach targeting efficiency in HEK293 cells (as judged by homology-directed “repair” of an integrated defective GFP gene) was achieved in 10-20% of cells through the use of I-Scel (Szczepek et al. Nature Biotechnology 2007; 25(7): 786-793).

A preferred class of meganucleases is the LAGLIDADG (SEQ ID NO: 2) endonucleases. These include I-Scel, I-Chul, I-Cre I, Csml, PI-Scel, PI-Tlil, PI-Mtul, I-Ceul, I-Scell, I-Scelll, HO, Pi-Civl, PI-Ctrl, PI-Aael, PI-Bsul, PI-Dhal, PI-Dral, PI-Mavl, PI-Mchl, PI-Mfu, PI-Mfll, PI-Mgal, PI-Mgol, PI-Minl, PI-Mkal, PI-Miel, PI-Mrnal, PI-Mshl, PI-Msml, PI-Mthl, PI-Mtu, PI-Mxel, PI-Npul, PI-Pful, PI-Rmal, PI-Spbl, PI-Sspl, PI-Facl, PI-Mjal, PI-Phol, Pi-Tagl, PI-Thyl, PI-Tko I, I-Msol, and PI-Tspl; preferably, I-Scel, I-Crel, I-Chul, I-Dmol, I-Csml, PI-Scel, PI-Pful, PI-Tlil, PI-Mtul, and I-Ceul.

In recent years a number of methods have been developed which allow the design of novel sequence-specific nucleases by fusing sequence-specific DNA binding domains to non-specific nucleases to create designed sequence-specific nucleases directed through bespoke DNA binding domains. Binding specificity can be directed by engineered binding domains such as zinc finger domains. These are small modular domains, stabilized by zinc ions, which are involved in molecular recognition and are used in nature to recognize DNA sequences. Arrays of zinc finger domains have been engineered for sequence specific binding and have been linked to the non-specific DNA cleavage domain of the type II restriction enzyme Fok1 to create zinc finger nucleases (ZFNs). Such ZFNs are preferred site-specific nucleases herein. ZFNs can be used to create double stranded break at specific sites within the genome. Fok1 is an obligate dimer and requires two ZFNs to bind in close proximity to effect cleavage. The specificity of engineered nucleases has been enhanced and their toxicity reduced by creating two different Fok1 variants which are engineering to only form heterodimers with each other [Doyon et al. Nat Methods 2011; 8(1): 74-79]. Such obligate heterodimer ZFNs have been shown to achieve homology-directed integration in 5-18% of target cells without the need for drug selection (Moehle et al. PNAS 2007; 104(9): 3055-3060, Perez-Pinera et al. Nucleic Acids Research 2012; 40(8): 3741-3752, Umov et al. Nature 2005; 435(7042): 646-651). Incorporation of inserts up to 8 kb with frequencies of >5% have been demonstrated in the absence of selection.

The ability to engineer DNA binding domains of defined specificity has been further simplified by the discovery in Xanthomonas bacteria of Transcription activator-like effectors (TALE) molecules. These TALE molecules consist of arrays of monomers of 33-35 amino acids with each monomer recognising a single base within a target sequence (Bogdanove et al. Science 2011; 333(6051): 1843-1846). This modular 1:1 relationship has made it relatively easy to design engineered TALE molecules to bind any DNA target of interest. By coupling these designed TALEs to Fok1 it has been possible to create novel sequence-specific TALE-nucleases. TALE nucleases, also known as TALENs, are preferred site-specific nucleases in this application and have been designed to a large number of sites (i.e. recognition sequences) and exhibit high success rate for efficient gene modification activity [Reyon et al. Nature Biotechnology 2012; 30(5): 460-465). Other variations and enhancements of TALE nuclease technology have been developed and could be used as site-specific nucleases in methods as described herein such as methods for generating a library. These included “mega-TALENs” where a TALE nuclease binding domain is fused to a meganuclease [Boissel et al. Nucleic Acids Research 2013; 42(4): 2591-2601] and “compact TALENs” where a single TALE nuclease recognition domain is used to effect cleavage (Beurdeley et al. Nature Communications 2013; 4:1762).

In recent years another system for directing double- or single-stranded breaks to specific sequences in the genome has been described. This system called “Clustered Regularly Interspaced Short Palindromic Repeats (CRISPR) and CRISPR Associated (Cas)” system is based on a bacterial defence mechanism (Sampson & Weiss. BioEssays: News and Reviews in Molecular, Cellular and Developmental Biology 2014; 36(1): 34-38). The CRISPR/Cas system is a preferred site-specific nuclease in a method for identifying a locus or in a method for generating a library. The CRISPR/Cas system targets DNA for cleavage via a short, complementary single-stranded RNA (CRISPR RNA or crRNA) adjoined to a short palindromic repeat. In the commonly used “Type II” system, the processing of the targeting RNA is dependent on the presence of a trans-activating crRNA (tracrRNA) that has sequence complementary to the palindromic repeat. Hybridization of the tracrRNA to the palindromic repeat sequence triggers processing. The processed RNA activates the Cas9 domain and directs its activity to the complementary sequence within DNA. The system has been simplified to direct Cas9 cleavage from a single RNA transcript and has been directed to many different sequences within the genome (Shalem et al. Science 2014; 343(6166): 84-87; Wang et al. Science 2014; 343(6166): 80-84). This approach to genome cleavage has the advantage of being directed via a short RNA sequence making it relatively simple to engineer cleavage specificity. Thus there are a number of different ways to achieve site-specific cleavage of genomic DNA. As described above this enhances the rate of integration of a donor plasmid through endogenous cellular DNA repair mechanisms.

In a method for generating a library as described herein, use of meganucleases, ZFNs, TALE nuclease or nucleic acid guided systems such as the CRISPR/Cas9 systems as site-specific nucleases will enable targeting of endogenous loci within the genome. Alternatively, in a method as described herein such as a method for generating a library, heterologous recognition sites (i.e. recognition sequences) for site-specific nucleases, including meganucleases, ZFNs and TALE nucleases could be introduced in advance. Nuclease-directed targeting could be used to drive insertion of recognition sequences by homologous recombination or NHEJ using vector DNA or even double stranded oligonucleotides (Orlando et al. Nucleic Acids Research 2010; 38(15): e152). As an alternative, non-specific targeting methods could be used to introduce recognition sequences for site-specific nucleases through the use of transposon-directed integration (Cadinanos & Bradley. Nucleic Acids Research 2007; 35(12): e87). Viral-based systems, such as lentivirus, applied at low titre could also be used to introduce recognition sequences.

The site-specific nuclease may be encoded by a single gene that is introduced on one plasmid, whereas the donor DNA is present on a second plasmid. Of course, combinations could be used incorporating two or more of these elements on the same plasmid and this could enhance the efficiency of targeting by reducing the number of number of plasmids to be introduced in a method for identifying a locus or a method for generating a library. In addition it may be possible to pre-integrate the nuclease(s) which could also be inducible to allow temporal control of nuclease activity as has been demonstrated for transposases (Cadinanos & Bradley. Nucleic Acids Research 2007; 35(12): e87). Finally the nuclease could be introduced as recombinant protein or protein: RNA complex (for example in the case of an RNA directed nuclease such as CRISPR: Cas9).

### Recognition Sequences

As noted, a method for generating a library involves providing a site-specific nuclease which cleaves a recognition sequence in cellular DNA.

In some embodiments, the recognition sequence is in the AAVS locus, as described for example in WO2015/166272, incorporated herein by reference in its entirety.

In some embodiments, the recognition sequence is in the ROSA26 locus, as described for example in Perez-Pinera et al. Nucleic Acids Research 2012; 40(8), incorporated herein by reference in its entirety.

Other suitable recognition sequences are described in WO2023/025834, incorporated herein by reference in its entirety. In some embodiments, the recognition sequence is in a neurolysin (NLN) gene, as described for example in WO2023/025834, incorporated herein by reference in its entirety. The eukaryotic cells used may contain endogenous sequences recognized by the site-specific nuclease or the recognition sequence may be engineered into the cellular DNA as earlier described herein. The neurolysin gene (human sequence: Uniprot Q9BYT8, ENSEMBL gene id ENSG00000123213) encodes a member of the metallopeptidase M3 protein family that cleaves neurotensin at the Pro10-Tyr11 bond, leading to the formation of neurotensin (1-10) and neurotensin (11-13). An exemplary sequence of a neurolysin gene is represented by SEQ ID NO: 3. In some embodiments, the recognition sequence is in a nucleic acid molecule represented by a nucleotide sequence comprising, consisting essentially of, or consisting of SEQ ID NO: 1, or a nucleotide sequence having at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identity with SEQ ID NO: 3.

In some embodiments, the recognition sequence is in a TRAF2 and NCK interacting kinase (TNIK) gene (Uniprot Q9UKE5, ENSEMBL gene id ENSG00000154310), as described for example in WO2023/025834, incorporated herein by reference in its entirety. An exemplary sequence of a TNIK gene is represented by SEQ ID NO: 4. In some embodiments, the recognition sequence is in a protein mono-ADP-ribosyltransferase 11 (PARP11) gene (Uniprot Q9NR21, ENSEMBL gene id ENSG00000111224), as described for example in WO2023/025834, incorporated herein by reference in its entirety. An exemplary sequence of a PARP11 gene is represented by SEQ ID NO: 5. In some embodiments, the recognition sequence is in a RAB40B gene (member RAS oncogene family, Uniprot Q12829, ENSEMBL gene id ENSG00000141542), as described for example in WO2023/025834, incorporated herein by reference in its entirety. An exemplary sequence of a RAB40B gene is represented by SEQ ID NO: 6. In some embodiments, the recognition sequence is in an abl interactor 2 (ABI2) gene (Uniprot Q9NYB9, ENSEMBL gene id ENSG00000138443), as described for example in WO2023/025834, incorporated herein by reference in its entirety. An exemplary sequence of an ABI2 gene is represented by SEQ ID NO: 7. In some embodiments, the recognition sequence is in a ring finger protein 19B (RNF19B) gene (Uniprot Q6ZMZ0, ENSEMBL gene id ENSG00000116514), as described for example in WO2023/025834, incorporated herein by reference in its entirety. An exemplary sequence of an RNF19B gene is represented by SEQ ID NO: 8. In some embodiments, the recognition sequence is in a CAMP-dependent protein kinase inhibitor alpha (PKIA) gene (Uniprot P61925, ENSEMBL gene id ENSG00000171033), as described for example in WO2023/025834, incorporated herein by reference in its entirety. An exemplary sequence of a PKIA gene is represented by SEQ ID NO: 9. In some embodiments, the recognition sequence is in a formimidoyltransferase cyclodeaminase (FTCD) gene (Uniprot O95954, ENSEMBL gene id ENSG00000160282), as described for example in WO2023/025834, incorporated herein by reference in its entirety. An exemplary sequence of an FTCD gene is represented by SEQ ID NO: 10.

In some embodiments, the recognition sequence is in an NLN gene, a TNIK gene or a RAB40B gene. In some embodiments, the recognition sequence is in a nucleic acid molecule represented by a nucleotide sequence comprising, consisting essentially of, or consisting of SEQ ID NOs: 3-10, or a nucleotide sequence having at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identity with SEQ ID NO: 3-10. In some embodiments, the recognition sequence is in an intron of a gene selected from an NLN, TNIK, PARP11, RAB40B, ABI2, RNF19B, PKIA, or FTCD gene, preferably an NLN, TNIK, or RAB40B genes, as described for example in WO2023/025834, incorporated herein by reference in its entirety. The term “intron” is used herein as customarily and ordinarily understood by the skilled person.

A recognition sequence in an intron of NLN is preferably in NLN-207 intron 1 (intron 1-2), intron 2 (intron 2-3) or intron 6 (intron 6-7). A recognition sequence in an intron of TNIK is preferably in TNIK-04 (Ensembl ID ENST00000436636.7) intron 2 (intron 2-3). A recognition sequence in an intron of PARP11 is preferably in PARP11-205 (Ensembl ID ENST00000450737.2) intron 1 (intron 1-2). A recognition sequence in an intron of RAB40B is preferably in RAB40B-206 (Ensembl ID ENST00000571995.6) intron 1 (intron 1-2). A recognition sequence in an intron of ABI2 is preferably in ABI2-203 (Ensembl ID ENST00000261018.12) intron 1 (intron 1-2). A recognition sequence in an intron of RNF19B is preferably in RNF19B-201 (Ensembl ID ENST00000235150.5) intron 1 (intron 1-2). A recognition sequence in an intron of PKIA is preferably in PKIA-202 (Ensembl ID ENST00000396418.7) intron 1 (intron 1-2). A recognition sequence in an intron of FTCD is preferably in FTCDNL1-201 (Ensembl ID ENST00000416668.5) intron 3 (intron 3-4).

In preferred embodiments, the recognition sequence is in an intron of a neurolysin gene. The canonical transcript of the human neurolysin (NLN) gene is NLN-201 (Ensembl transcript ID: ENST00000380985.10) which comprises 13 exons. An alternative transcript is NLN-207 (Ensembl transcript ID: ENST00000509935.2) which comprises 7 exons. In some embodiments, the recognition sequence is in NLN-201 intron 1 of a neurolysin gene (NLN-201 intron 1-2; exemplary sequence: SEQ ID NO: 11). In some embodiments, the recognition sequence is in NLN-201 intron 2 of a neurolysin gene (NLN-201 intron 2-3; exemplary sequence: SEQ ID NO: 12). In some embodiments, the recognition sequence is in NLN-201 intron 3 of a neurolysin gene (NLN-201 intron 3-4; exemplary sequence: SEQ ID NO: 13). In some embodiments, the recognition sequence is in NLN-201 intron 4 of a neurolysin gene (NLN-201 intron 4-5; exemplary sequence: SEQ ID NO: 14). In some embodiments, the recognition sequence is in NLN-201 intron 5 of a neurolysin gene (NLN-201 intron 5-6; exemplary sequence: SEQ ID NO: 15). In some embodiments, the recognition sequence is in NLN-201 intron 6 of a neurolysin gene (NLN-201 intron 6-7; exemplary sequence: SEQ ID NO: 16). In some embodiments, the recognition sequence is in NLN-201 intron 7 of a neurolysin gene (NLN-201 intron 7-8; exemplary sequence: SEQ ID NO: 17). In some embodiments, the recognition sequence is in NLN-201 intron 8 or NLN-207 intron 1 of a neurolysin gene (NLN-201 intron 8-9 or NLN-207 intron 1-2; exemplary sequence: SEQ ID NO: 18). In some embodiments, the recognition sequence is in NLN-201 intron 9 or NLN-207 intron 2 of a neurolysin gene (NLN-201 intron 9-10 or NLN-207 intron 2-3; exemplary sequence: SEQ ID NO: 19). In some embodiments, the recognition sequence is in NLN-201 intron 10 or NLN-207 intron 3 of a neurolysin gene (NLN-201 intron 10-11 or NLN-207 intron 3-4; exemplary sequence: SEQ ID NO: 20). In some embodiments, the recognition sequence is in NLN-201 intron 11 or NLN-207 intron 4 of a neurolysin gene (NLN-201 intron 11-12 or NLN-207 intron 4-5; exemplary sequence: SEQ ID NO: 21). In some embodiments, the recognition sequence is in intron 12 of a NLN-201 neurolysin gene (NLN-201 intron 12-13; exemplary sequence: SEQ ID NO: 22). In some embodiments, the recognition sequence is in intron 5 of a NLN-207 neurolysin gene (NLN-207 intron 5-6; exemplary sequence: SEQ ID NO: 23). In some embodiments, the recognition sequence is in intron 6 of a NLN-207 neurolysin gene (NLN-207 intron 6-7; exemplary sequence: SEQ ID NO: 24).

Preferred introns are NLN-207 introns 1, 2, and 6 of an NLN gene. In some embodiments, the recognition sequence is in a nucleic acid molecule represented by a nucleotide sequence comprising, consisting essentially of, or consisting of SEQ ID NOs: 18, 19, 24, or a nucleotide sequence having at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identity with SEQ ID NOs: 18, 19, 24.

In some embodiments, particularly when the recognition sequence is in an intron of a gene selected from an NLN, TNIK, PARP11, RAB40B, ABI2, RNF19B, PKIA, or FTCD gene as described above, the recognition sequence is in an open chromatin region of the intron.

In some embodiments, particularly when the recognition sequence is in an intron of a gene selected from an NLN, TNIK, PARP11, RAB40B, ABI2, RNF19B, PKIA, or FTCD gene as described above, the recognition sequence is in an enhancer region of the intron.

As used herein “open chromatin” or “euchromatin” or “loose chromatin” refers to a structure that is permissible for transcription whereas “heterochromatin” or “tight” or “closed” chromatin is more compact and more refractory to factors that need to gain access to the DNA template.

### Distribution of Recognition Sequences

A recognition sequence for the site-specific nuclease in a method as described herein may be present in genomic DNA, or episomal DNA which is stably inherited in the cells. Donor DNA may therefore be integrated at a genomic or episomal locus in the cellular DNA.

In its simplest form a donor DNA is targeted to a single site within the eukaryotic genome. Identification of a cell demonstrating a particular binding activity or cellular phenotype will allow direct isolation of the gene encoding the desired property (e.g., by PCR from mRNA or genomic DNA). This is facilitated by using a unique recognition sequence for the site-specific nuclease, occurring once in the cellular DNA. Cells used for creation of the library may thus contain a nuclease recognition sequence at a single fixed locus, i.e., one identical locus in all cells. Libraries produced from such cells will contain donor DNA integrated at the fixed locus, i.e., occurring at the same locus in cellular DNA of all clones in the library.

Optionally, recognition sequences may occur multiple times in cellular DNA, so that the cells have more than one potential integration site for donor DNA. This would be a typical situation for diploid or polyploid cells where the recognition sequence is present at corresponding positions in a pair of chromosomes, i.e., replicate loci. Libraries produced from such cells may contain donor DNA integrated at replicate fixed loci. For example libraries produced from diploid cells may have donor DNA integrated at duplicate fixed loci and libraries produced from triploid cells may have donor DNA integrated at triplicate fixed loci. Many suitable mammalian cells are diploid, and clones of mammalian cell libraries as described herein may have donor DNA integrated at duplicate fixed loci.

The sequence recognised by the site-specific nuclease may occur at more than one independent locus in the cellular DNA. Donor DNA may therefore integrate at multiple independent loci. Libraries of diploid or polyploid cells may comprise donor DNA integrated at multiple independent fixed loci and/or at replicate fixed loci.

In cells containing recognition sequences at multiple loci (whether replicate or independent loci), each locus represents a potential integration site for a molecule of donor DNA. Introduction of donor DNA into the cells may result in integration at the full number of nuclease recognition sequences present in the cell, or the donor DNA may integrate at some but not all of these potential sites. For example, when producing a library from diploid cells containing recognition sequences at first and second fixed loci (e.g., duplicate fixed loci), the resulting library may comprise clones in which donor DNA is integrated at the first fixed locus, clones in which donor DNA is integrated at the second fixed locus, and clones in which donor DNA is integrated at both the first and second fixed loci.

Methods of producing libraries as described herein may therefore involve site-specific nuclease cleavage of multiple fixed loci in a cell, and integration of donor DNA at the multiple fixed loci. As noted above, in cases where there are multiple copies of the same recognition sequence (e.g., as occurs when targeting endogenous loci in diploid or polyploid cells) it is possible that nucleic acid sequences encoding a binder (or binder subunit) will be integrated, particularly when an efficient targeting mechanisms is used, with only one nucleic acid sequence encoding a binder (or binder subunit) being specific to the target. This can be resolved during subsequent screening once nucleic acid sequences encoding a binder (or binder subunit) have been isolated.

### Donor DNA

A method for generating a library as described herein may comprise integrating a donor DNA. Preferred donor DNA molecules are described in this section.

The donor DNA will usually be circularised DNA, and may be provided as a plasmid or vector. Linear DNA is another possibility. Donor DNA molecules may comprise regions that do not integrate into the cellular DNA, in addition to one or more donor DNA sequences that integrate into the cellular DNA. The DNA is typically double-stranded, although single-stranded DNA may be used in some cases. The donor DNA contains one or more transgenes encoding a binder, for example it may comprise a promoter: gene cassette.

In the simplest format double-stranded, circular plasmid DNA can be used to drive homologous recombination. This requires regions of DNA flanking the transgenes which are homologous to DNA sequence flanking the cleavage site in genomic DNA. Linearised double-stranded plasmid DNA or PCR product or synthetic genes could be used to drive both homologous recombination and NHEJ repair pathways. As an alternative to double-stranded DNA it is possible to use single-stranded DNA to drive homologous recombination (Fujioka et al. Nucleic Acids Res 1993; 21(3): 407-412). A common approach to generating single-stranded DNA is to include a single-stranded origin of replication from a filamentous bacteriophage into the plasmid.

Single-stranded DNA viruses such as adeno-associated virus (AAV) have been used to drive efficient homologous recombination where the efficiency has been shown to be improved by several orders of magnitude (Khan et al. Nat Prot 2011; 6(4):482-501); Deyle & Russell. Current Opinion in Molecular Therapeutics 2009; 11(4):442-447). Systems such as the AAV systems could be used in conjunction with nuclease-directed cleavage in a method for identifying a locus and in a method for generating a library.

The benefits of both systems could be applied to in a method for identifying a locus and in a method for generating a library. The packaging limit of AAV vectors is 4.7 kb but the use of nuclease digestion of target genomic DNA will reduce this allowing larger transgene constructs to be incorporated.

A molecule of donor DNA may encode a single binder or multiple binders. Optionally, multiple subunits of a binder may be encoded per molecule of donor DNA. In some embodiments, donor DNA encodes a subunit of a multimeric binder.

Promoters in Donor DNA and Selection of Clones with Integrated Donor DNA

In a method as described herein such as a method for generating a library, the donor DNA comprises one or more nucleic acid sequences encoding a binding domain coupled to an Fc domain. Transcription of the binder or binder subunit from the encoding donor DNA will usually be achieved by placing the sequence encoding the binder or binder subunit under control of a promoter and optionally one or more enhancer elements for transcription. A promoter (and optionally other genetic control elements) may be included in the donor DNA molecule itself. Alternatively, the sequence encoding the binder may lack a promoter on the donor DNA, and instead may be placed in operable linkage with a promoter on the cellular DNA, e.g., an endogenous promoter or a pre-integrated exogenous promoter, as a result of its insertion at the integration site created by the site-specific nuclease.

In some embodiments, the donor DNA molecules as described herein comprise a first promoter operably linked to the first nucleic acid sequence and/or a second promoter operably linked to the second nucleic acid sequence.

In some embodiments, the first and second promoter are the same. In preferred embodiments, the first and second promoter are different. In some embodiments, each of the first and second promoter may independently be a constitutive or an inducible promoter, preferable a constitutive promoter. Suitable constitutive promoters include a CMV promoter, a CAG promoter and an EF1alpha promoter, or variants thereof. Preferred promoters are a CMV and an EF1alpha promoter. A more preferred promoter is a CMV promoter. Thus, each of the first and second promoter may independently be selected from the group consisting of a CMV promoter, a CAG promoter and an EF1alpha promoter, preferably from the group consisting of a CMV promoter and an EF1alpha promoter.

In preferred embodiments, the first promoter is a CMV promoter and the second promoter is an EF1alpha promoter, or the first promoter is an EF1alpha promoter and the second promoter is a CMV promoter. More preferably, the first promoter is a CMV promoter and the second promoter is an EF1alpha promoter.

In some embodiments, the donor DNA molecules as described herein comprise a bidirectional promoter operably linked to the first and second nucleic acid sequence. A preferred bidirectional promoter is a bidirectional CMV promoter.

Donor DNA may further comprise one or more further coding sequences, such as genetic elements enabling selection of cells containing or expressing the donor DNA. Such an element may be called a selectable marker. As with the sequence encoding the binder, discussed above, such elements may be associated with a promoter on the donor DNA or may be placed under control of a promoter as a result of integration of the donor DNA at a fixed locus. The latter arrangement provides a convenient means of selecting specifically for those cells which have integrated the donor DNA at the desired site, since these cells should express the genetic element for selection. This may be, for example, a gene conferring resistance to a negative selection agent such as blasticidin or puromycin. One or more selection steps may be applied to remove unwanted cells, such as cells that lack the donor DNA or which have not integrated the donor DNA at the correct position.

The expression of a membrane anchored binder could itself be used as a form of selectable marker. For example for libraries as described herein, ten cells which express the binder can be selected using secondary reagents which recognise the surface expressed Fc using methods described herein. Upon initial transfection with donor DNA encoding the binder under the control of an exogenous promoter, transient expression (and cell surface expression) of the binder will occur and it will be necessary to wait for transient expression to abate (to achieve targeted integration of e.g., 1-2 antibody genes/cell).

As an alternative a construct encoding a membrane tethering element or a membrane anchor (e.g., the Fc domain of the present example fused to the PDGF receptor transmembrane domain) could be pre-integrated before the binders sequences are introduced.

Accordingly, in some embodiments, methods as described herein are such that the first and/or second nucleic acid sequence as described herein further encodes a membrane anchor, such as a transmembrane domain or a membrane localization signal. A preferred transmembrane domain is the PDGF receptor transmembrane domain. A preferred membrane localization signal is a GPI recognition sequence. Preferably, a membrane anchor as described herein is fused to the binder (subunit), more preferably to the Fc domain.

In some embodiments, the first but not the second nucleic acid sequence further encodes a membrane anchor. In some embodiments, the second but not the first nucleic acid sequence further encodes a membrane anchor.

If this membrane-tethering element lacks a promoter or is encoded within an exon which is out of frame with the preceding exon then surface expression will be compromised. Targeted integration of an incoming donor molecule can then correct this defect (e.g., by targeting a promoter or an “in-frame” exon into the intron which is upstream of the defective tethering element). If the frame “correcting exon” also encodes a binder then a fusion will be produced between the binder and the membrane tethering element resulting in surface expression of both. Thus correctly targeted integration will result in-frame expression of the membrane tethering element alone or as part of a fusion with the incoming binder. Furthermore if the incoming library of binders lack a membrane tethering element and these are incorrectly integrated they will not be selected. Thus expression of the binder itself on the cell surface can be used to select the population of cells with correctly targeted integration.

### Number of Clones and Library Diversity

A method for generating a library as described herein is for generating a library of eukaryotic cell clones containing DNA encoding a diverse repertoire of bispecific binders. In the context of this application, a library refers to a library of eukaryotic cell clones containing DNA encoding a diverse repertoire of binders which may be obtained via one of these methods, unless explicitly mentioned otherwise. Preferred libraries and their properties are defined in this section.

Yeast display libraries of 10⁷-10¹⁰ have previously been constructed and demonstrated to yield binders in the absence of immunisation or pre-selection of the population (Chao et al. Nat Protoc 2006; 1(2): 755-768; Benatuil et al. Protein Engineering, Design and Selection 2010; 23(4): 155-159; Feldhaus et al. Nat Biotechnol 2003; 21:163-170; Zhao et al. Journal of Immunological Methods 2011; 363(2):221-232). Many of the previously published mammalian display libraries used antibody genes derived from immunised donors or even enriched antigen-specific B lymphocytes, given the limitations of library size and variability when using cells from higher eukaryotes. Thanks to the efficiency of gene targeting in the methods described herein large, naive libraries can be constructed in higher eukaryotes such as mammalian cells, which match those described for simpler eukaryotes such as yeast.

Following integration of donor DNA into the cellular DNA, the resulting recombinant cells are cultured to allow their replication, generating a clone of cells from each initially-produced recombinant cell. Each clone is thus derived from one original cell into which donor DNA was integrated at an integration site created by the site-specific nuclease. Methods as described herein are associated with a high efficiency and high fidelity of donor DNA integration, and a library as described herein may contain at least 100, 10³, 10⁴, 10⁵, 10⁶, 10⁷, 10⁸, 10⁹ or 10¹⁰ clones.

Without being bound to this theory, using nuclease-directed integration it is possible to target 10% or more of transfected mammalian cells. It is also practical to grow and transform >10¹⁰ cells (e.g. from 5 litres of cells growing at 2×10⁶ cells/ml). Transfection of such large numbers of cells could be done using standard methods including polyethyleneimine-mediated transfection as described herein. In addition methods are available for highly efficient electroporation of 10¹⁰ cells in 5 minutes e.g. http://www.maxcyte.com. Thus using the approach of the present disclosure it is possible to create libraries in excess of 10⁹ clones.

When the population of donor DNA molecules that is used to create the library contains multiple copies of the same sequence, two or more clones may be obtained that contain DNA encoding the same binder. It can also be the case that a clone may contain donor DNA encoding more than one different binder, for example if there is more than one recognition sequence for the site-specific nuclease, as detailed elsewhere herein. Thus, the diversity of the library, in terms of the number of different binders encoded or expressed, may be different from the number of clones obtained.

Clones in the library preferably contain donor DNA encoding one or two members of the repertoire of binders and/or preferably express only one or two members of the repertoire of binders. A limited number of different binders per cell is an advantage when it comes to identifying the clone and/or DNA encoding a particular binder identified when screening the library against a given target. This is simplest when clones encode a single member of the repertoire of binders. However it is also straightforward to identify the relevant encoding DNA for a desired binder if a clone selected from a library encodes a small number of different binders, for example a clone may encode two members of the repertoire of binders. As discussed elsewhere herein, clones encoding one or two binders are particularly convenient to generate by selecting a recognition sequence for the site-specific nuclease that occurs once per chromosomal copy in a diploid genome, as diploid cells contain duplicate fixed loci, one on each chromosomal copy, and the donor DNA may integrate at one or both fixed loci. Thus, clones of the library may each express only one or two members of the repertoire of binders.

Binders displayed on the surface of cells of the library may be identical to (having the same amino acid sequence as) other binders displayed on the same cell. The library may consist of clones of cells which each display a single member of the repertoire of binders, or of clones displaying a plurality of members of the repertoire of binders per cell. Alternatively a library may comprise some clones that display a single member of the repertoire of binders, and some clones that display a plurality of members (e.g., two) of the repertoire of binders.

Accordingly, a library as described herein may comprise clones encoding more than one member of the repertoire of binders, wherein the donor DNA is integrated at duplicate fixed loci or multiple independent fixed loci.

As noted above, it is easiest to identify the corresponding encoding DNA for a binder if the corresponding clone expresses only one binder. Typically, a molecule of donor DNA will encode a single binder. The binder may be multimeric so that a molecule of donor DNA includes multiple genes or open reading frames corresponding to the various subunits of the multimeric binder.

A library as described herein may encode at least 100, 10³, 10⁴, 10⁵ or 10⁶, 10⁷, 10⁸, 10⁹ or 10¹⁰ different binders. Where the binders are multimeric, diversity may be provided by one or more subunits of the binder. Multimeric binders may combine one or more variable subunits with one or more constant subunits, where the constant subunits are the same (or of more limited diversity) across all clones of the library. In generating libraries of multimeric binders, combinatorial diversity is possible where a first repertoire of binder subunits may pair with any of a second repertoire of binder subunits.

### Characteristics and Form of the Library

Methods as described herein enable construction of eukaryotic cell libraries having many advantageous characteristics. The libraries preferably have any one or more of the following features:
1. Diversity. A library may encode and/or express at least 100, 10³, 10⁴, 10⁵, 10⁶, 10⁷, 10⁸ or 10⁹ different binders.
2. Uniform integration. A library may consist of clones containing donor DNA integrated at a fixed locus, or at a limited number of fixed loci in the cellular DNA. Each clone in the library therefore contains donor DNA at the fixed locus or at least one of the fixed loci. Preferably clones contain donor DNA integrated at one or two fixed loci in the cellular DNA. As explained elsewhere herein, the integration site is at a recognition sequence for a site-specific nuclease. Integration of donor DNA to produce recombinant DNA is described in detail elsewhere herein and can generate different results depending on the number of integration sites. Where there is a single potential integration site in cells used to generate the library, the library will be a library of clones containing donor DNA integrated at the single fixed locus. All clones of the library therefore contain the binder genes at the same position in the cellular DNA. Alternatively where there are multiple potential integration sites, the library may be a library of clones containing donor DNA integrated at multiple and/or different fixed loci. Preferably, each clone of a library contains donor DNA integrated at a first and/or a second fixed locus. For example a library may comprise clones in which donor DNA is integrated at a first fixed locus, clones in which donor DNA is integrated at a second fixed locus, and clones in which donor DNA is integrated at both the first and second fixed loci. In preferred embodiments there are only one or two fixed loci in the clones in a library, although it is possible to integrate donor DNA at multiple loci if desired for particular applications. Therefore in some libraries each clone may contain donor DNA integrated at any one or more of several fixed loci, e.g., three, four, five or six fixed loci. For libraries containing binder subunits integrated at separate sites, clones of the library may contain DNA encoding a first binder subunit integrated at a first fixed locus and DNA encoding a second binder subunit integrated at a second fixed locus, wherein the clones express multimeric binders comprising the first and second subunits.
3. Uniform transcription. Relative levels of transcription of the binders between different clones of the library is kept within controlled limits due to donor DNA being integrated at a controlled number of loci, and at the same locus in the different clones (fixed locus). Relatively uniform transcription of binder genes leads to comparable levels of expression of binders on or from clones in a library. Binders displayed on the surface of cells of the library may be identical to (having the same amino acid sequence as) other binders displayed on the same cell. The library may consist of clones of cells which each display a single member of the repertoire of binders, or of clones displaying a plurality of members of the repertoire of binders per cell. Alternatively a library may comprise some clones that display a single member of the repertoire of binders, and some clones that display a plurality of members (e.g., two) of the repertoire of binders. Preferably clones of a library express one or two members of the repertoire of binders. For example, a library of eukaryotic cell clones according to the present disclosure may express a repertoire of at least 10³, 10⁴, 10⁵ 10⁶, 10⁷, 10⁸ or 10⁹ different binders, each cell containing donor DNA integrated at a fixed locus in the cellular DNA. The donor DNA encodes the binder and may further comprise a genetic element for selection of cells into which the donor DNA is integrated at the fixed locus. Cells of the library may contain DNA encoding an exogenous site-specific nuclease.

These and other features of libraries are further described elsewhere herein.

The present disclosure extends to the library either in pure form, as a population of library clones in the absence of other eukaryotic cells, or mixed with other eukaryotic cells. Other cells may be eukaryotic cells of the same type (e.g., the same cell line) or different cells. Further advantages may be obtained by combining two or more libraries as described herein, or combining a library as described herein with a second library or second population of cells, either to facilitate or broaden screening or for other uses as are described herein or which will be apparent to the skilled person.

A library as described herein, one or more clones obtained from the library, or host cells into which DNA encoding a binder from the library has been introduced, may be provided in a cell culture medium. The cells may be cultured and then concentrated to form a cell pellet for convenient transport or storage.

Libraries as described herein will usually be provided in vitro. The library may be in a container such as a cell culture flask containing cells of the library suspended in a culture medium, or a container comprising a pellet or concentrated suspension of eukaryotic cells comprising the library. The library may constitute at least 75%, 80%, 85% or 90% of the eukaryotic cells in the container.

It is understood that the fixed locus where the donor DNA is integrated for the libraries as described herein corresponds with the location of the recognition sequence of methods for generating a library as described herein. Thus, all preferences for the location of the recognition sequences as described herein are also applicable to the fixed locus of the library as described herein.

### Binder Display

A library built in the context of this disclosure may be cultured to express the bispecific binders in either soluble secreted form or in transmembrane form, preferably in transmembrane form. It is said that the expressed bispecific binders are “displayed” if they are retained on the surface of the cells which encode them. In this context, terms like “binder display”, “display on/at the surface”, “display on the cell”, “display of the binder” and the like may be used interchangeably. In this context, a library may also be called a display or a display library.

Preferably, a library wherein the expressed bispecific binders are displayed is to provide a repertoire of bispecific binders for screening against a target of interest.

Bispecific binders may comprise or be linked to a membrane anchor, such as a transmembrane domain, for extracellular display of the binder at the cell surface. This may involve direct fusion of the binder, i.e. one or both of its subunits, to a membrane localisation signal such as a GPI recognition sequence or to a transmembrane domain such as the transmembrane domain of the PDGF receptor, as described elweshere herein (Gronwald et al. Proc Natl Acad Sci USA 1988; 85(10):3435-9). Retention of binders at the cell surface can also be done indirectly by association with another cell surface retained molecule expressed within the same cell. This associated molecule could itself be part of a heterodimeric binder, such as tethered antibody heavy chain in association with a light chain partner that is not directly tethered.

Although cell surface immobilisation facilitates selection of the binder, in many applications it is necessary to prepare cell-free, secreted binder. It will be possible to combine membrane tethering and soluble secretion using a recapture method of attaching the secreted binders to cell surface receptors. One approach is to format the library of binders as secreted molecules which can associate with a membrane anchored molecule expressed within the same cell which can function to capture a secreted binder. For example, in the case of antibodies or binder molecules fused to antibody Fc domains, a membrane tethered Fc can “sample” secreted binder molecules being expressed in the same cell resulting in display of a monomeric fraction of the binder molecules being expressed while the remainder is secreted in a bivalent form (U.S. Pat. No. 8,551,715). An alternative is to use a tethered IgG binding domain such as protein A.

Other methods for retaining secreted antibodies with the cells producing them are reviewed in Kumar et al. (Methods 2012; 56(3), 366-374) and include encapsulation of cells within microdrops, matrix aided capture, affinity capture surface display (ACSD), secretion and capture technology (SECANT) and “cold capture”. In examples given for ACSD and SECANT, biotinylation is used to facilitate immobilisation of streptavidin or a capture antibody on the cell surface. The captured molecule in turn captures secreted antibodies. In the example of SECANT in vivo biotinylated of the secreted molecule occurs. Using the “cold capture” technique secreted antibody can be detected on producer cells using antibodies directed to the secreted molecule. It has been proposed that this due to association of the secreted antibody with the glycocalyx of the cell [86]. Alternatively it has been suggested that the secreted product is trapped by staining antibodies on the cell surface before being endocytosed [87]. The above methods have been used to identify high expressing clones within a population but could potentially be adapted for identification of binding specificity, provided the association has sufficient longevity at the cell surface.

Even when the binder is directly tethered to the cell surface it is possible to generate a soluble product. For example the gene encoding the selected binder can be recovered and cloned into an expression vector lacking the membrane anchored sequence. Alternatively, an expression construction can be used in which the transmembrane domain is encoded within an exon flanked by recombination sites, e.g., ROX recognition sites for Ore recombinase (Anastassiadis et al. Disease Models & Mechanisms 2009; 2(9-10): 508-515). The exon encoding the transmembrane domain can be removed by transfection with a gene encoding Dre recombinase to switch expression to a secreted form.

Any of the above methods or other suitable approaches can be used to ensure that bispecific binders expressed by clones of a library are displayed on the surface of their expressing cells.

### Derivative Libraries

Following a method for producing a library as described herein, one or more library clones may be selected and used to produce a further, second generation library. When a library has been generated by introducing DNA into eukaryotic cells as described herein, the library may be cultured to express the bispecific binders, and one or more clones expressing bispecific binders of interest may be recovered, for example by selecting bispecific binders against a target, optionally two targets, via a method for identifying a bispecific binder to a target as described herein. These clones may subsequently be used to generate a derivative library containing DNA encoding a second repertoire of bispecific binders, preferably via a method for producing a library as described herein.

To generate the derivative library, DNA of the one or more recovered clones is mutated to provide the second repertoire of bispecific binders. Mutations may be addition, substitution or deletion of one or more nucleotides. Where the binder is a polypeptide, mutation will be to change the sequence of the encoded binder by addition, substitution or deletion of one or more amino acids. Mutation may be focussed on one or more regions, such as one or more CDRs of an antibody molecule, providing a repertoire of bispecific binders of a common structural class which differ in one or more regions of diversity, as described elsewhere herein.

Generating the derivative library may comprise isolating DNA from the one or more recovered clones, introducing mutation into the DNA to provide a derivative population of donor DNA molecules encoding a second repertoire of bispecific binders, and introducing the derivative population of donor DNA molecules into cells to create a derivative library of cells containing DNA encoding the second repertoire of bispecific binders.

Isolation of the DNA may involve obtaining and/or identifying the DNA from the clone. Such methods may encompass amplifying the DNA encoding a bispecific binder from a recovered clone, e.g., by PCR and introducing mutations. DNA may be sequenced and mutated DNA synthesised.

Mutation may alternatively be introduced into the DNA in the one or more recovered clones by inducing mutation of the DNA within the clones. The derivative library may thus be created from one or more clones without requiring isolation of the DNA, e.g., through endogenous mutation in avian DT40 cells.

Antibody display lends itself especially well to the creation of derivative libraries. Once antibody genes are isolated, it is possible to use a variety of mutagenesis approaches (e.g., error prone PCR, oligonucleotide-directed mutagenesis, chain shuffling) to create display libraries of related clones from which improved variants can be selected. For example, with chain-shuffling the DNA encoding the population of selected VH clone, oligoclonal mix or population can be sub-cloned into a vector encoding a suitable antibody format and encoding a suitably formatted repertoire of VL chains (Dyson et al. Anal Biochem 2011; 417(1): 25-35). Alternatively and again using the example of VHS, the VH clone, oligomix or population could be introduced into a population of eukaryotic cells which encode and express a population of appropriately formatted light chain partners (e.g., a VL-CL chain for association with an IgG or Fab formatted heavy chain). The VH population could arise from any of the sources discussed above including B cells of immunised animals or scFv genes from selected phage populations. In the latter example cloning of selected VHs into a repertoire of light chains could combine chain shuffling and re-formatting (e.g., into IgG format) in one step.

A particular advantage of display on eukaryotic cells is the ability to control the stringency of the selection/screening step. By reducing antigen concentration, cells expressing the highest affinity binders can be distinguished from lower affinity clones within the population. The visualisation and quantification of the affinity maturation process using flow cytometry is a major benefit of eukaryotic display as it gives an early indication of percentage positives in naive library and allows a direct comparison between the affinity of the selected clones and the parental population during sorting. Following sorting, the affinity of individual clones can be determined by pre-incubating with a range of antigen concentrations and analysis in flow cytometry or with a homogenous Time Resolved Fluorescence (TRF) assay or using surface plasmon resonance (SPR) (Biacore).

### Screening to Identify or Select Binders to a Target of Interest

As noted, the eukaryotic cell library may be used in a method of screening for a bispecific binder that recognises a target of interest, optionally two targets of interest. Accordingly, an an aspect, there is provided a use of a library as described herein as a display library for selecting bispecific binders to a target of interest, optionally to two targets of interest.

Such a method may comprise:
providing a library via the method for producing a library as described herein, or
providing a library as described herein,
culturing cells of the library to express the bispecific binders,
exposing the bispecific binders to the target, optionally the two targets, allowing
recognition of the target, optionally of the two targets, by one or more binders of interest, if present, and
detecting whether the target, optionally the two targets, is recognised by a binder of interest.

A method according to this aspect may be called a method for identifying a binder to a target in the context of this application. In this context, the selection of binder or the screening for a binder also refer to such a method.

Methods for identifying a bispecific binder to a target may be carried out using a range of target molecule classes, e.g., protein, nucleic acid, carbohydrate, lipid, small molecules. The target may be provided in soluble form. The target may be labelled to facilitate detection, e.g., it may carry a fluorescent label or it may be biotinylated. Cells expressing a target-specific binder may be isolated using a directly or indirectly labelled target molecule, where the binder captures the labelled molecule. For example, cells that are bound, via the binder: target interaction, to a fluorescently labelled target can be detected and sorted by flow cytometry or FACS to isolate the desired cells. Selections involving cytometry require target molecules which are directly fluorescently labelled or are labelled with molecules which can be detected with secondary reagents, e.g., biotinylated target can be added to cells and binding to the cell surface can be detected with fluorescently labelled streptavidin such as streptavidin-phycoerythrin. A further possibility is to immobilise the target molecule or secondary reagents which bind to the target on a solid surface, such as magnetic beads or agarose beads, to allow enrichment of cells which bind the target. For example cells that bind, via the binder: target interaction, to a biotinylated target can be isolated on a substrate coated with streptavidin, e.g., streptavidin-coated beads.

In libraries used in methods for identifying a bispecific binder to a target it is preferable to over-sample, i.e., screen more clones than the number of independent clones present within the library to ensure effective representation of the library. Identifying bispecific binders from very large libraries provided by this disclosure could be done by flow sorting but this would take several days, particularly if over-sampling the library. As an alternative initial selections could be based on the use of recoverable antigen, e.g., biotinylated antigen recovered on streptavidin-coated magnetic beads. Thus streptavidin-coated magnetic beads could be used to capture cells which have bound to biotinylated antigen. Selection with magnetic beads could be used as the only selection method or this could be done in conjunction with flow cytometry where better resolution can be achieved, e.g., differentiating between a clone with higher expression levels and one with a higher affinity (Feldhaus et al. Nat Biotechnol 2003; 21(2): 163-170; Zhao et al. Journal of Immunological Methods 2011; 363(2): 221-232).

The in vitro nature of display technology approaches makes it is possible to control selection in a way that is not possible by immunisation, e.g., selecting on a particular conformational state of a target (Biffi et al. Nature Chemistry 2013; 5(3): 182-186; Gao et al. PNAS 2009; 106(9):3071-3076). Targets could be tagged through chemical modification (fluorescein, biotin) or by genetic fusion (e.g. protein fused to an epitope tag such as a FLAG tag or another protein domain or a whole protein). The tag could be nucleic acid (e.g., DNA, RNA or non-biological nucleic acids) where the tag is part fused to target nucleic acid or could be chemically attached to another type of molecule such as a protein. This could be through chemical conjugation or through enzymatic attachment (Gu et al. Nat Biotechnol 2013; 30(2):144-152). Nucleic acid could be also fused to a target through a translational process such as ribosome display. The “tag” may be another modification occurring within the cell (e.g., glycosylation, phosphorylation, ubiqitinylation, alkylation, PASylation, SUMO-lation and others described at the Post-translational Database (db-PTM) at https://awi.cuhk.edu.cn/dbPTM, see Li et al. Nucleic Acids Research 2022; 50(D1): D471-479) which can be detected via secondary reagents. This would yield binders which bind an unknown target protein on the basis of a particular modification.

Targets could be detected using existing binders which bind to that target molecule, e.g., target specific antibodies. Use of existing binders for detection will have the added advantage of identifying binders within the library of binders which recognise an epitope distinct from the binder used for detection. In this way pairs of binders could be identified for use in applications such as sandwich ELISA. Where possible a purified target molecule would be preferred. Alternatively the target may be displayed on the surface of a population of target cells and the binders are displayed on the surface of the library cells, the method comprising exposing the binders to the target by bringing the library cells into contact with the target cells. Recovery of the cells expressing the target (e.g., using biotinylated cells expressing target) will allow enrichment of cells which express binders to them. This approach would be useful where low affinity interactions are involved since there is the potential for a strong avidity effect.

The target molecule could also be unpurified recombinant or unpurified native targets provided a detection molecule is available to identify cell binding (as described above).

In addition binding of target molecules to the cell expressing the bispecific binder could be detected indirectly through the association of target molecule to another molecule which is being detected, e.g., a cell lysate containing a tagged molecule could be incubated with a library of binders to identify binders not only to the tagged molecule but also binders to its associated partner proteins. This would result in a panel of antibodies to these partners which could be used to detect or identify the partner (e.g., using mass spectrometry). Cellular fractionation could be used to enrich targets from particular sub-cellular locations. Alternatively differential biotinylation of surface or cytoplasmic fractions could be used in conjunction with streptavidin detection reagents for eukaryotic display (Cho & Shusta. Protein Eng Des Sel 2010; 23(7):567-577). The use of detergent solubilised target preparations is a particularly useful approach for intact membrane proteins such as GPCRs and ion channels which are otherwise difficult to prepare. The presence of detergents may have a detrimental effect on the eukaryotic cells displaying the binders requiring recovery of binder genes without additional growth of the selected cells.

Following detection of target recognition by a binder of interest, cells of a clone containing DNA encoding the binder of interest may be recovered. DNA encoding the binder may then be isolated (e.g., identified or amplified) from the recovered clone, thereby obtaining DNA encoding a binder that recognises the target. Optionally, the DNA encoding a binder that recognises the target may be sequenced.

Exemplary binders and targets are detailed elsewhere herein. A classic example is a library of bispecific antibody molecules, which may be screened for binding to a target antigen of interest, optionally two target antigens of interest.

### Phenotype Screens

In a preferred method for identifying a bispecific binder to a target (or optionally two targets) the binder is able to modify cell signaling and/or cellular behavior as a result of the action of the bispecific binder on the target(s). In more preferred methods, the bispecific binder is a bispecific antibody.

Thus, the eukaryotic cell library may be used in a method of screening for a cell of a desired phenotype, wherein the phenotype results from expression of a bispecific binder by the cell. Accordingly, an an aspect, there is provided a use of a library as described herein for screening for cells displaying a desired cellular phenotype, wherein the phenotype results from expression of a bispecific binder by a cell.

Such a method may comprise:
providing a library via the method for producing a library as described herein, or
providing a library as described herein;
culturing cells of the library to express the bispecific binders; and
detecting whether the desired phenotype is exhibited.

A method according to this aspect may be called a method for screening for a cell of a desired phenotype in the context of this application.

Bispecific antibodies which modify cell signalling by binding to ligands or receptors have a proven track record in drug development and the demand for such therapeutic antibodies continues to grow. Such antibodies and other classes of functional bispecific binders also have potential in controlling cell behaviour in vivo and in vitro. The ability to control and direct cellular behaviour however relies on the availability of natural ligands which control specific signalling pathways. Unfortunately many natural ligands such as those controlling stem cell differentiation (e.g., members of FGF, TGF-beta, Wnt and Notch super-families) often exhibit promiscuous interactions and have limited availability due to their poor expression/stability profiles. Due to their specificity, bispecific antibodies have great potential in controlling cellular behaviour.

The identification of functional antibodies that modify cell signalling has historically been relatively laborious involving picking clones, expressing antibody, characterising according to sequence and binding properties, conversion to mammalian expression systems and addition to functional cell based assays. The eukaryotic display approach described herein will reduce this effort but there is still a requirement for production of antibody and addition to a separate reporter cell culture. Therefore, a preferred alternative may be to directly screen libraries of bispecific binders expressed in eukaryotic cells for the effect of binding on cell signalling or cell behaviour by using the production cell itself as a reporter cell. Following introduction of antibody genes, clones within the resulting population of cells showing alteration in reporter gene expression or altered phenotypes can be identified.

A number of recent publications have described the construction of antibody libraries by cloning repertoires of antibody genes into reporter cells (Zhang et al. Chemistry & Biology 2013; 20(5), 734-741; Melidoni et al. Proceedings of the National Academy of Sciences 2013; 110(44), 17802-1780; Zhang et al. Proceedings of the National Academy of Sciences 2012; 109(39), 15728-15733). These systems combine expression and reporting within one cell, and typically introduce a population of antibodies selected against a pre-defined target (e.g., using phage display).

A population of antibody genes may be introduced into reporter cells to produce a library by methods described herein, and clones within the population with an antibody-directed alteration in phenotype (e.g., altered gene expression or survival) can be identified. For this phenotypic-directed selection to work there is a requirement to retain a linkage between the antibody gene present within the expressing cell (genotype) and the consequence of antibody expression (phenotype). This has been achieved previously either through tethering the antibody to the cell surface (Zhang et al. Chemistry & Biology 2013; 20(5), 734-741) as described for antibody display or through the use of semi-solid medium to retain secreted antibodies in the vicinity of producing cells (Melidoni et al. Proceedings of the National Academy of Sciences 2013; 110(44), 17802-1780). Alternatively antibodies and other binders can be retained inside the cell (Xie et al. Chemistry & Biology 2014; 2(2), 274-283).

Binders retained on the cell surface or in the surrounding medium can interact with an endogenous or exogenous receptor on the cell surface causing activation of the receptor. This in turn can cause a change in expression of a reporter gene or a change in the phenotype of the cell. As an alternative the antibody can block the receptor or ligand to reduce receptor activation. The gene encoding the binder which causes the modified cellular behaviour can then be recovered for production or further engineering. As an alternative to this “target-directed” approach, it is possible to introduce a “naive” antibody population which has not been pre-selected to a particular target (Yea et al. PNAS 2013; 110:14966-14971). The cellular reporting system is used to identify members of the population with altered behaviour. Since there is no prior knowledge of the target, this non-targeted approach has a particular requirement for a large antibody repertoire, since pre-enrichment of the antibody population to the target is not possible. This approach will benefit from using nuclease-directed transgene integration as described herein.

The “functional selection” approach could be used on other applications involving libraries in eukaryotic cells, particularly higher eukaryotes such as mammalian cells. The antibody could be fused to a signalling domain such that binding to target causes activation of the receptor. Kawahara et al. have constructed chimeric receptors where an extra-cellular scFv targeting fluorescein was fused to a spacer domain (the D2 domain of the Epo receptor) and various intracellular cytokine receptor domain including the thrombopoeitin (Tpo) receptor, erythropoietin (Epo) receptor, gp130, IL-2 receptor and the EGF receptor (Kawahara et al. Biochem Biophys Res Commun 2004; 315(1): 132-138; Sogo et al. Cytokine 2009; 46(1): 127-139; Kawahara et al. Cytokine 2011; 55(3): 402-408). These were introduced into an IL-3 dependent proB cell line (BaF3) (Palacios & Steinmetz. Cell 1985; 41(3): 727-734), where chimaeric receptors were shown to exhibit antigen-dependent activation of the chimaeric receptor leading to IL-3 independent growth. This same approach was used in model experiments to demonstrate antigen mediated chemoattraction of BaF3 cells [110]. The approach was extended beyond stable culture cells to primary cells exemplified by the survival and growth of Tpo-responsive haematopoeitic stem cells (Kawahara et al. Cytokine 2011; 55(3): 402-408) or IL2 dependant primary T cells where normal stimulation by Tpo and IL-2 respectively was replaced by fluorescein directed stimulation of scFv chimaeric receptors. Thus a system based on chimaeric antibody-receptor chimaeras can be used to drive target dependent gene expression or phenotypic changes in primary or stable reporter cells. This capacity could be used to identify fused binders which drive a signalling response or binders which inhibit the response.

In a modification of the above approach separate VH and VL domains from an anti-lysozyme antibody were fused to the Epo intracellular domain [Ueda et al. J Immunol Methods 2000; 241(1-2): 159-170). Cells grew in response to addition of lysozyme indicating an antigen induced dimerisation or stabilisation of the separate VH and VL fusion partners. Thus three interacting components come together for an optimal response in this system.

Although described here with reference to antibody molecules, the above methods for identifying a binder (i.e. an antibody molecule) to a target may also be adapted and performed with libraries of other types of binders.

Protein fragment complementation represents an alternative system for studying and for selecting protein:protein interactions in mammalian cells (Kerppola. Chemical Society Reviews 2009; 38(10): 2876-2886, Michnick et al. Nature Reviews Drug Discovery 2007; 6(7): 569/582). This involves restoring function of split reporter proteins through protein:protein interactions. Reporter proteins which have been used include ubiquitin, DNAE intein, beta-galactosidase, dihydrofolate reductase, GFP, firefly luciferase, beta-lactamase, TEV protease. For example a recent example of this approach is the mammalian membrane 2 hybrid (MaMTH) approach where association of a bait protein: split ubiquitin: transcription factor fusion with a partner protein: split ubiquitin restores ubiquitin recognition and liberates the transcription factor to effect reporter gene expression (Petschnigg et al. Nat Methods 2014; 11(5): 585-92). Again binders which interfere with or enhance this interaction could be identified through perturbed signalling.

Following detection of a desired phenotype, cells of a clone that exhibits the desired phenotype may then be recovered. DNA encoding the binder may then be isolated (e.g., identified or amplified) from the recovered clone, thereby obtaining DNA encoding a bispecific binder which produces the desired phenotype when expressed in the cell. Optionally, the DNA encoding a binder that recognises the target may be sequenced.

When the desired phenotype is detected, cells of a clone that exhibits the desired phenotype may then be recovered. Optionally, DNA encoding the bispecific binder is then isolated from the recovered clone, providing DNA encoding a bispecific binder which produces the desired phenotype when expressed in the cell. Optionally, the DNA encoding a bispecific binder which produces the desired phenotype may be sequenced.

### Recovery and Reformatting of Binders and Encoding DNA

After a binder is identified via a method for identifying a binder to a target, a common next step will be to isolate (e.g., identify or amplify) the DNA encoding the binder. Optionally, it may be desired to modify the nucleic acid encoding the binder, for example to restructure the binder and/or to insert the encoding sequence into a different vector. Hence, a preferred method for identifying a binder to a target comprises isolating the DNA encoding the binder recognizing the target. More preferred methods are described below.

Where the binder is an antibody molecule, a preferred method for identifying a binder to a target comprises isolating DNA encoding the antibody molecule from cells of a clone, amplifying DNA encoding at least one antibody variable region, preferably both the VH and VL domain, and inserting DNA into a vector to provide a vector encoding the antibody molecule.

Additional aspects and embodiments of the disclosure are set out in the following numbered paragraphs which form an integral part of the description and may be appropriately combined with aspects and embodiments described elsewhere herein.
1. A method for producing a library of eukaryotic cell clones containing DNA encoding a diverse repertoire of bispecific binders, the method comprising:
   providing eukaryotic cells;
   providing a plurality of donor DNA molecules, each donor DNA molecule comprising a first nucleic acid sequence encoding a first binding domain coupled to a first Fc domain; and a second nucleic acid sequence encoding a second binding domain coupled to a second Fc domain;
   introducing the donor DNA into the cells; and
   culturing the recombinant cells to produce clones, thereby providing a library of eukaryotic cell clones containing DNA encoding the repertoire of bispecific binders.
2. A method for producing a library of eukaryotic cell clones containing DNA encoding a diverse repertoire of multimeric, e.g. dimeric, bispecific binders, the method comprising:
   providing eukaryotic cells containing DNA encoding a first subunit of the bispecific binders, the DNA comprising a first nucleic acid sequence encoding a first binding domain coupled to a first Fc domain;
   providing a plurality of donor DNA molecules encoding a second subunit of the bispecific binders, each donor DNA molecule comprising a second nucleic acid sequence encoding a second binding domain coupled to a second Fc domain;
   introducing the donor DNA into the cells, thereby creating recombinant cells which contain donor DNA integrated in the cellular DNA; and
   culturing the recombinant cells to produce clones containing DNA encoding the first and second subunits, thereby providing a library of eukaryotic cell clones containing DNA encoding the repertoire of bispecific binders.
3. A method for producing a library of eukaryotic cell clones containing DNA encoding a diverse repertoire of multimeric, e.g. dimeric, bispecific binders, the method comprising:
   providing eukaryotic cells;
   providing a plurality of first donor DNA molecules encoding a first subunit of the bispecific binders, each donor DNA molecule comprising a first nucleic acid sequence encoding a first binding domain coupled to a first Fc domain;
   introducing the first donor DNA into the cells to create a first set of recombinant cells containing the first donor DNA integrated in the cellular DNA;
   culturing the first set of recombinant cells to produce a first set of clones containing DNA encoding the first subunit;
   providing a plurality of second donor DNA molecules encoding a second subunit of the bispecific binders, each donor DNA molecule comprising a second nucleic acid sequence encoding a second binding domain coupled to a second Fc domain;
   introducing the second donor DNA into cells of the first set of clones to create a second set of recombinant cells containing the first and second donor DNA integrated in the cellular DNA; and
   culturing the second set of recombinant cells to produce a second set of clones containing DNA encoding the first and second subunits, thereby providing a library of eukaryotic cell clones containing DNA encoding the repertoire of bispecific binders.
4. A method for producing a library of eukaryotic cell clones containing DNA encoding a diverse repertoire of multimeric, e.g. dimeric, bispecific binders, the method comprising:
   providing eukaryotic cells;
   providing a plurality of first donor DNA molecules encoding a first subunit of the bispecific binders, each donor DNA molecule comprising a first nucleic acid sequence encoding a first binding domain coupled to a first Fc domain;
   providing a plurality of second donor DNA molecules encoding a second subunit of the bispecific binders, each donor DNA molecule comprising a second nucleic acid sequence encoding a second binding domain coupled to a second Fc domain;
   introducing the first and second donor DNA into the cells to create recombinant cells containing the first and second donor DNA integrated in the cellular DNA; and
   culturing the recombinant cells to produce clones, thereby providing a library of eukaryotic cell clones containing donor DNA encoding the repertoire of bispecific antibodies.
5. A method according to any one of the preceding paragraphs, wherein the first and second Fc domains are engineered to promote heterodimerization.
6. A method according to any one of the preceding paragraphs, wherein one or more of the step(s) of introducing the donor DNA into the cells comprises providing a site-specific nuclease within the cells, wherein the nuclease cleaves a recognition sequence in cellular DNA to create an integration site at which the donor DNA becomes integrated into the cellular DNA, integration occurring through DNA repair mechanisms endogenous to the cells, thereby creating recombinant cells containing donor DNA integrated in the cellular DNA.
7. A method according to paragraph 6, wherein the recognition sequence is in an NLN gene, a TNIK gene or a RAB40B gene.
8. A method according to paragraph 6 or 7, wherein the recognition sequence is in an NLN gene.
9. A method according to any one of paragraphs 6-8, wherein the recognition sequence is in an intron of a gene.
10. A method according to any one of paragraphs 6-9, wherein the recognition sequence is in an open chromatin region of an intron.
11. A method according to any one of paragraphs 6-10, wherein the recognition sequence is in an enhancer region of an intron.
12. A method according to any one of paragraphs 6-11, wherein the recognition sequence is in NLN-207 intron 1, 2 or 6 of the NLN gene.
13. A method according to any one of the preceding paragraphs, wherein the bispecific binders are bispecific antibody molecules.
14. A method according to any one of the preceding paragraphs, wherein the bispecific binders are multimeric, for example dimeric, comprising at least a first and a second subunit.
15. A method according to any one of the preceding paragraphs,
wherein the first and/or second binding domain comprises a single antibody variable domain, preferably a variable domain derived from a heavy-chain antibody such as a VHH or a V_{NAR}, and/or
wherein the first and/or second binding domain comprises two antibody variable domains, preferably a single-chain variable fragment (scFv).
16. A method according to any one of the preceding paragraphs, wherein the first binding domain comprises a single antibody variable domain, preferably a variable domain derived from a heavy-chain antibody such as a VHH or a V_{NAR}, more preferably a VHH.
17. A method according to any one of the preceding paragraphs, wherein the second binding domain comprises two antibody variable domains, preferably a single-chain variable fragment (scFv).
18. A method according to any one of the preceding paragraphs, wherein the first Fc domain comprises a knob mutation and the second Fc domain comprises a hole mutation, or wherein the first Fc domain comprises a hole mutation and the second Fc domain comprises a knob mutation.
19. A method according to any one of the preceding paragraphs, wherein the donor DNA molecules are flanked by homology arms.
20. A method according to any one of the preceding paragraphs, wherein the donor DNA molecules comprise a first promoter operably linked to the first nucleic acid sequence and/or wherein the donor DNA molecules comprise a second promoter operably linked to the second nucleic acid sequence.
21. A method according to any one of the preceding paragraphs, wherein the donor DNA molecules comprise a bidirectional promoter operably linked to the first and second nucleic acid sequence.
22. A method according to any one of the preceding paragraphs, wherein the first and/or second nucleic acid sequence encodes a membrane anchor, such as a transmembrane domain or a membrane localization signal.
23. A method according to any one of the preceding paragraphs, wherein the repertoire of bispecific binders is a plurality of polypeptides which share a common structure and have one or more regions of amino acid sequence diversity.
24. A method according to any one of the preceding paragraphs, wherein the repertoire of bispecific binders is a repertoire of bispecific antibody molecules differing in one or more complementarity determining regions.
25. A method according to any one of the preceding paragraphs, wherein the bispecific binder further comprises one or more additional subunits, which may be introduced on the same donor DNA as the first or second subunit or which may be integrated at separate sites in the cellular DNA.
26. A method according to any one of the preceding paragraphs, wherein the cells are higher eukaryotic cells with a genome size of greater than 2×10\hat{ }7 base pairs.
27. A method according to any one of the preceding paragraphs, wherein the cells are mammalian, avian, insect or plant cells.
28. A method according to paragraph 27, wherein the cells are mammalian cells, preferably wherein the cells are human cells.
29. A method according to paragraph 28, wherein the cells are HEK293 cells, Chinese hamster ovary (CHO) cells, T lymphocyte lineage cells or B lymphocyte lineage cells or any of the cell lines listed in the “Cancer Cell Line Encyclopedia” or “COSMIC catalogue of somatic mutations in cancer”.
30. A method according to paragraph 29, wherein the cells are primary T cells or a T cell line.
31. A method according to paragraph 29, wherein the cells are primary B cells, a B cell line, a pre-B cell line or a pro-B cell line.
32. A method according to paragraph 31, wherein the cells are murine pre-B cell line 1624-5, IL-3 dependent pro-B cell line Ba/F3 or chicken DT40 B cells.
33. A method according to any one of paragraphs 6-32, wherein the recognition sequence for the site-specific nuclease is in genomic DNA of the cells.
34. A method according to any one of paragraphs 6-33, wherein the recognition sequence for the site-specific nuclease is in episomal DNA within the cells.
35. A method according to any one of paragraphs 6-34, wherein the recognition sequence for the site-specific nuclease occurs only once or twice in the cellular DNA.
36. A method according to any one of paragraphs 6-35, wherein the site-specific nuclease cleaves cellular DNA to create a double strand break serving as an integration site.
37. A method according to any one of paragraphs 6-36, wherein the nuclease is a meganuclease.
38. A method according to any one of paragraphs 6-37, wherein the nuclease is a zinc finger nuclease (ZFN).
39. A method according to any one of paragraphs 6-38, wherein the nuclease is a TALE nuclease.
40. A method according to any one of paragraphs 6-39, wherein the nuclease is a nucleic acid guided nuclease.
41. A method according to paragraph 40, wherein DNA cleavage is directed by the CRISPR/Cas system.
42. A method according to any one of the preceding paragraphs, wherein the donor DNA is integrated into the cellular DNA by homologous recombination.
43. A method according to any one of the preceding paragraphs, wherein the donor DNA is integrated into the genomic DNA by non-homologous end joining or microhomology-directed end joining.
44. A method according to any one of the preceding paragraphs, wherein the donor DNA comprises a genetic element for selection of cells into which the donor DNA is integrated.
45. A method according to any one of the preceding paragraphs, wherein integration of the donor DNA into the cellular DNA places expression of the bispecific binder or a subunit thereof and/or expression of a genetic selection element under control of a promoter present within the cellular DNA.
46. A method according to any one of the preceding paragraphs, wherein the donor DNA comprises a sequence encoding the bispecific binder or a subunit thereof operably linked to a promoter.
47. A method according to any one of the preceding paragraphs, wherein the library contains at least 100, 10\hat{ }3, 10\hat{ }4, 10\hat{ }5, 10\hat{ }6, 10\hat{ }7, 10\hat{ }8 or 10\hat{ }9 clones, each clone being derived from an individual recombinant cell produced by integration of donor DNA.
48. A method according to any one of the preceding paragraphs, wherein the library encodes at least 100, 10\hat{ }3, 10\hat{ }4, 10\hat{ }5, 10\hat{ }6, 10\hat{ }7, 10\hat{ }8 or 10\hat{ }9 different bispecific binders.
49. A method according to any one of the preceding paragraphs, wherein each clone contains integrated donor DNA encoding only one or two members of the repertoire of bispecific binders.
50. A method according to any one of the preceding paragraphs, wherein the eukaryotic cells are diploid and contain a recognition sequence for the site-specific nuclease at duplicate fixed loci in the cellular DNA.
51. A method according to any one of the preceding paragraphs, wherein each clone contains integrated donor DNA encoding a single member of the repertoire of bispecific binders.
52. A method according to any one of the preceding paragraphs, wherein the donor DNA molecules each encode a single bispecific binder or subunit thereof.
53. A method according to any one of the preceding paragraphs, wherein the bispecific binders are displayed on the cell surface.
54. A method according to any one of the preceding paragraphs, wherein the bispecific binders are secreted from the cells.
55. A method according to any one of the preceding paragraphs, further comprising: culturing the library to express the bispecific binders,
recovering one or more clones expressing a bispecific binder of interest, and
generating a derivative library from the one or more recovered clones, wherein the derivative library contains DNA encoding a second repertoire of bispecific binders.
56. A method according to paragraph 55, wherein generating the derivative library comprises isolating donor DNA from the one or more recovered clones, introducing mutation into the DNA to provide a derivative population of donor DNA molecules encoding a second repertoire of bispecific binders, and introducing the derivative population of donor DNA molecules into cells to create a derivative library of cells containing DNA encoding the second repertoire of bispecific binders.
57. A method according to paragraph 55, wherein generating the derivative library comprises introducing mutation into the donor DNA in the one or more recovered clones by inducing mutation of the DNA within the clones.
58. A method of producing a diverse repertoire of bispecific binders, comprising producing a library by a method according to any one of the preceding paragraphs and culturing the library cells to express the bispecific binders.
59. A library produced by a method according to any one of the preceding paragraphs.
60. A method of screening for a cell of a desired phenotype, wherein the phenotype results from expression of a bispecific binder by the cell, the method comprising
   providing a library via the method for producing a library according to any one of the preceding paragraphs, or providing a library according to paragraph 59;
   culturing cells of the library to express the bispecific binders; and
   detecting whether the desired phenotype is exhibited.
61. A method according to paragraph 60, wherein the phenotype is expression of a reporter gene in a cell that expresses the binder.
62. A method according to paragraph 60 or paragraph 61, further comprising recovering cells of a clone that expresses a bispecific binder that produces the desired phenotype.
63. A method according to paragraph 62, further comprising isolating DNA encoding the bispecific binder from the recovered clone, thereby obtaining DNA encoding a bispecific binder which produces the desired phenotype.
64. A method of screening for a bispecific binder that recognises a target of interest, said method comprising:
   providing a library via the method for producing a library according to any one of paragraphs 1 to 58, or providing a library according to paragraph 59;
   culturing cells of the library to express the bispecific binders;
   exposing the bispecific binders to the target, optionally the two targets, allowing recognition of the target, optionally of the two targets, by one or more binders of interest, if present; and
   detecting whether the target, optionally the two targets, is recognised by a binder of interest.
65. A method according to paragraph 64, wherein the target is provided in soluble form.
66. A method according to paragraph 64, wherein the target is displayed on the surface of a population of target cells and the bispecific binders are displayed on the surface of the library cells, the method comprising exposing the bispecific binders to the target by bringing the library cells into contact with the target cells.
67. A method according to any one of paragraphs 60 to 66, wherein the bispecific binders are bispecific antibody molecules and the target is an antigen.
68. A method according to any one of paragraphs 64 to 67 further comprising detecting target recognition by a bispecific binder of interest, and recovering cells of a clone containing DNA encoding the bispecific binder of interest.
69. A method according to paragraph 68, further comprising isolating DNA encoding the bispecific binder from the recovered clone, thereby obtaining DNA encoding a bispecific binder that recognises the target.
70. A method according to paragraph 63 or paragraph 69, comprising introducing mutation or converting the DNA to modified DNA encoding a restructured bispecific binder.
71. A method according to paragraph 70, wherein the bispecific binder comprises an scFv and the method comprises converting DNA encoding the scFv to DNA encoding an Ig or fragment thereof while maintaining the original variable VH and VL chain pairings.
72. A method according to paragraph 63, 69, 70 or 71, further comprising introducing the DNA into a host cell.
73. An in vitro library of eukaryotic cell clones that express a diverse repertoire of at least 100, 10\hat{ }3, 10\hat{ }4, 10\hat{ }5, 10\hat{ }6, 10\hat{ }7, 10\hat{ }8 or 10\hat{ }9 different bispecific binders, each cell containing recombinant DNA wherein donor DNA encoding a bispecific binder or subunit thereof is integrated at least a first and/or a second fixed locus in the cellular DNA.
74. A library according to paragraph 59 or 73 wherein the clones also contain DNA comprising a genetic element for selection of cells that express the encoded bispecific binder.
75. A library according to paragraph 59, 73 or 74 wherein the bispecific binders are antibody molecules.
76. A container containing eukaryotic cells comprising a library according to any one of paragraphs 59 or 73 to 75.
77. A container according to paragraph 76, wherein the library constitutes at least 75%, 80%, 85% or 90% of the eukaryotic cells in the container.
78. A container according to paragraph 76 or 77, wherein the container is a cell culture flask containing cells of the library suspended in a culture medium.
79. A container according to paragraph 76 or paragraph 77, comprising a pellet or concentrated suspension of eukaryotic cells comprising the library.
80. Use of a site-specific nuclease for targeted cleavage of cellular DNA in the construction of a library of eukaryotic cells containing DNA encoding a repertoire of bispecific binders, wherein nuclease-mediated DNA cleavage enhances site-specific integration of binder genes through endogenous cellular DNA repair mechanisms.
81. Use of the library of any one of paragraphs 59 or 73 to 75 as a display library for selecting bispecific binders to a desired target.
82. Use of the library of any one of paragraphs 59 or 73 to 75 for screening for cells displaying a desired cellular phenotype, wherein the phenotype results from expression of a bispecific binder by a cell.

### General Information

Unless stated otherwise, all technical and scientific terms used herein have the same meaning as customarily and ordinarily understood by a person of ordinary skill in the art to which this disclosure belongs, and read in view of this disclosure.

As used herein, the term “promoter” or “regulatory sequence” refers to a nucleic acid fragment that functions to control the transcription of one or more coding sequences, and is located upstream with respect to the direction of transcription of the transcription initiation site of the coding sequence, and is structurally identified by the presence of a binding site for DNA-dependent RNA polymerase, transcription initiation sites and any other DNA sequences, including, but not limited to transcription factor binding sites, repressor and activator protein binding sites, and any other sequences of nucleotides known to one of skill in the art to act directly or indirectly to regulate the amount of transcription from the promoter. A “constitutive” promoter is a promoter that is active in most tissues under most physiological and developmental conditions. An “inducible” and/or “repressible” promoter is a promoter that is physiologically or developmentally regulated to be induced and/or repressed, e.g. by the application of a chemical inducer or repressing signal.

As used herein, the term “operably linked” refers to a linkage of polynucleotide elements in a functional relationship. A nucleic acid is “operably linked” when it is placed into a functional relationship with another nucleic acid sequence. For instance, a transcription regulatory sequence such as a promoter is operably linked to a coding sequence if it affects the transcription of the coding sequence. Operably linked means that the DNA sequences being linked are typically contiguous and, where necessary to join two protein encoding regions, contiguous and in reading frame.

The terms “protein” or “polypeptide” are used interchangeably and refer to molecules consisting of a chain of amino acids, without reference to a specific mode of action, size, 3-dimensional structure or origin.

### Sequence Identity

It is to be understood that each nucleic acid molecule or protein fragment or polypeptide or peptide or derived peptide or construct as identified herein by a given sequence identity number (SEQ ID NO) is not limited to this specific sequence as disclosed. Each coding sequence as identified herein encodes a given protein fragment or polypeptide or peptide or derived peptide or construct or is itself a protein fragment or polypeptide or construct or peptide or derived peptide.

Throughout this application, each time one refers to a specific nucleotide sequence SEQ ID NO (take SEQ ID NO: X as example) encoding a given protein fragment or polypeptide or peptide or derived peptide, one may replace it by:
i. a nucleotide sequence comprising a nucleotide sequence that has at least 60% sequence identity with SEQ ID NO: X;
ii. a nucleotide sequence the sequence of which differs from the sequence of a nucleic acid molecule of (i) due to the degeneracy of the genetic code; or
iii. a nucleotide sequence that encodes an amino acid sequence that has at least 60% amino acid identity or similarity with an amino acid sequence encoded by a nucleotide sequence SEQ ID NO: X.

Another preferred level of sequence identity or similarity is 65%. Another preferred level of sequence identity or similarity is 70%. Another preferred level of sequence identity or similarity is 75%. Another preferred level of sequence identity or similarity is 80%. Another preferred level of sequence identity or similarity is 85%. Another preferred level of sequence identity or similarity is 90%. Another preferred level of sequence identity or similarity is 95%. Another preferred level of sequence identity or similarity is 97%. Another preferred level of sequence identity or similarity is 99%. Another preferred level of sequence identity or similarity is 99.5%.

Throughout this application, each time one refers to a specific amino acid sequence SEQ ID NO (take SEQ ID NO: Y as example), one may replace it by: a polypeptide represented by an amino acid sequence comprising a sequence that has at least 60% sequence identity or similarity with amino acid sequence SEQ ID NO: Y. Another preferred level of sequence identity or similarity is 65%. Another preferred level of sequence identity or similarity is 70%. Another preferred level of sequence identity or similarity is 75%. Another preferred level of sequence identity or similarity is 80%. Another preferred level of sequence identity or similarity is 85%. Another preferred level of sequence identity or similarity is 90%. Another preferred level of sequence identity or similarity is 95%. Another preferred level of sequence identity or similarity is 97%. Another preferred level of sequence identity or similarity is 99%. Another preferred level of sequence identity or similarity is 99.5%.

Each nucleotide sequence or amino acid sequence described herein by virtue of its identity or similarity percentage with a given nucleotide sequence or amino acid sequence respectively has in a further preferred embodiment an identity or a similarity of at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5% or 100% with the given nucleotide or amino acid sequence, respectively.

Each non-coding nucleotide sequence (i.e. of a promoter or of another regulatory region) could be replaced by a nucleotide sequence comprising a nucleotide sequence that has at least 60% sequence identity or similarity with a specific nucleotide sequence SEQ ID NO (take SEQ ID NO: A as example). A preferred nucleotide sequence has at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5% or 100% identity with SEQ ID NO: A. In a preferred embodiment, such non-coding nucleotide sequence such as a promoter exhibits or exerts at least an activity of such a non-coding nucleotide sequence such as an activity of a promoter as known to a person of skill in the art.

The terms “homology”, “sequence identity” and the like are used interchangeably herein. Sequence identity is described herein as a relationship between two or more amino acid (polypeptide or protein) sequences or two or more nucleic acid (polynucleotide) sequences, as determined by comparing the sequences. In a preferred embodiment, sequence identity is calculated based on the full length of two given SEQ ID NO's or on a part thereof, preferably based on the full length. Part thereof preferably means at least 50%, 60%, 70%, 80%, 90%, or 100% of both SEQ ID NO's. In the art, “identity” also refers to the degree of sequence relatedness between amino acid or nucleic acid sequences, as the case may be, as determined by the match between strings of such sequences. “Similarity” between two amino acid sequences is determined by comparing the amino acid sequence and its conserved amino acid substitutes of one polypeptide to the sequence of a second polypeptide. “Identity” and “similarity” can be readily calculated by known methods, including but not limited to those described in Bioinformatics and the Cell: Modern Computational Approaches in Genomics, Proteomics and transcriptomics, Xia X., Springer International Publishing, New York, 2018; and Bioinformatics: Sequence and Genome Analysis, Mount D., Cold Spring Harbor Laboratory Press, New York, 2004, each incorporated herein by reference. “Sequence identity” and “sequence similarity” can be determined by alignment of two peptide or two nucleotide sequences using global or local alignment algorithms, depending on the length of the two sequences. Sequences of similar lengths are preferably aligned using a global alignment algorithm (e.g. Needleman-Wunsch) which aligns the sequences optimally over the entire length, while sequences of substantially different lengths are preferably aligned using a local alignment algorithm (e.g. Smith-Waterman). Sequences may then be referred to as “substantially identical” or “essentially similar” when they (when optimally aligned by for example the program EMBOSS needle or EMBOSS water using default parameters) share at least a certain minimal percentage of sequence identity (as described below).

A global alignment is suitably used to determine sequence identity when the two sequences have similar lengths. When sequences have a substantially different overall length, local alignments, such as those using the Smith-Waterman algorithm, are preferred. EMBOSS needle uses the Needleman-Wunsch global alignment algorithm to align two sequences over their entire length (full length), maximizing the number of matches and minimizing the number of gaps. EMBOSS water uses the Smith-Waterman local alignment algorithm. Generally, the EMBOSS needle and EMBOSS water default parameters are used, with a gap open penalty=10 (nucleotide sequences)/10 (proteins) and gap extension penalty=0.5 (nucleotide sequences)/0.5 (proteins). For nucleotide sequences the default scoring matrix used is DNAfull and for proteins the default scoring matrix is Blosum62 (Henikoff & Henikoff, 1992, PNAS 89, 915-919, incorporated herein by reference).

Alternatively percentage similarity or identity may be determined by searching against public databases, using algorithms such as FASTA, BLAST, etc. Thus, the nucleic acid and protein sequences of some embodiments of the present disclosure can further be used as a “query sequence” to perform a search against public databases to, for example, identify other family members or related sequences. Such searches can be performed using the BLASTn and BLASTx programs (version 2.0) of Altschul, et al. (1990) J. Mol. Biol. 215:403-10, incorporated herein by reference. BLAST nucleotide searches can be performed with the NBLAST program, score=100, wordlength=12 to obtain nucleotide sequences homologous to nucleic acid molecules of the disclosure. BLAST protein searches can be performed with the BLASTx program, score=50, wordlength=3 to obtain amino acid sequences homologous to protein molecules of the disclosure. To obtain gapped alignments for comparison purposes, Gapped BLAST can be utilized as described in Altschul et al., (1997) Nucleic Acids Res. 25(17): 3389-3402, incorporated herein by reference. When utilizing BLAST and Gapped BLAST programs, the default parameters of the respective programs (e.g., BLASTx and BLASTn) can be used. See the homepage of the National Center for Biotechnology Information accessible on the world wide web at www.ncbi.nlm.nih.gov/.

Optionally, in determining the degree of amino acid similarity, the skilled person may also take into account so-called conservative amino acid substitutions. As used herein, “conservative” amino acid substitutions refer to the interchangeability of residues having similar side chains. Examples of classes of amino acid residues for conservative substitutions are given in the Tables below.

| | |
|---|---|
| Acidic Residues | Asp (D) and Glu (E) |
| Basic Residues | Lys (K), Arg (R), and His (H) |
| Hydrophilic Uncharged Residues | Ser (S), Thr (T), Asn (N), and Gln (Q) |
| Aliphatic Uncharged Residues | Gly (G), Ala (A), Val (V), Leu (L), and |
| | Ile (I) |
| Non-polar Uncharged Residues | Cys (C), Met (M), and Pro (P) |
| Aromatic Residues | Phe (F), Tyr (Y), and Trp (W) |

Alternative conservative amino acid residue substitution classes:

| | | | |
|---|---|---|---|
| 1 | A | S | T |
| 2 | D | E | |
| 3 | N | Q | |
| 4 | R | K | |
| 5 | I | L | M |
| 6 | F | Y | W |

Alternative physical and functional classifications of amino acid residues:

| | |
|---|---|
| Alcohol group-containing residues | S and T |
| Aliphatic residues | I, L, V, and M |
| Cycloalkenyl-associated residues | F, H, W, and Y |
| Hydrophobic residues | A, C, F, G, H, I, L, M, R, T, V, W, and Y |
| Negatively charged residues | D and E |
| Polar residues | C, D, E, H, K, N, Q, R, S, and T |
| Positively charged residues | H, K, and R |
| Small residues | A, C, D, G, N, P, S, T, and V |
| Very small residues | A, G, and S |
| Residues involved in turn formation | A, C, D, E, G, H, K, N, Q, R, S, P and T |
| Flexible residues | Q, T, K, S, G, P, D, E, and R |

For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulphur-containing side chains is cysteine and methionine. Preferred conservative amino acids substitution groups are: valine-leucine-isoleucine, phenylalanine-tyrosine, lysine-arginine, alanine-valine, and asparagine-glutamine. Substitutional variants of the amino acid sequence disclosed herein are those in which at least one residue in the disclosed sequences has been removed and a different residue inserted in its place. Preferably, the amino acid change is conservative. Preferred conservative substitutions for each of the naturally occurring amino acids are as follows: Ala to Ser; Arg to Lys; Asn to Gln or His; Asp to Glu; Cys to Ser or Ala; Gln to Asn; Glu to Asp; Gly to Pro; His to Asn or Gln; Ile to Leu or Val; Leu to Ile or Val; Lys to Arg; Gln or Glu; Met to Leu or Ile; Phe to Met, Leu or Tyr; Ser to Thr; Thr to Ser; Trp to Tyr; Tyr to Trp or Phe; and, Val to Ile or Leu.

In this document and in its claims, the verb “to comprise” and its conjugations is used in its non-limiting sense to mean that items following the word are included or contained, but items not specifically mentioned are not excluded. Thus, the terms ‘comprising’, ‘comprises’, ‘comprised of’ and the like as used herein are synonymous with ‘including’, ‘includes’ or ‘containing’, ‘contains’, and are inclusive or open-ended and do not exclude additional, non-recited members, elements or method steps.

In addition, the verb “to consist” may be replaced by “to consist essentially of” meaning that a composition as described herein may comprise additional component(s) than the ones specifically identified, said additional component(s) not altering the unique characteristic of this disclosure. In addition, the verb “to consist” may be replaced by “to consist essentially of” meaning that a method as described herein may comprise additional step(s) than the ones specifically identified, said additional step(s) not altering the unique characteristic of this disclosure.

As used herein, the singular forms ‘a’, ‘an’, and ‘the’ include both singular and plural referents unless the context clearly dictates otherwise; for example, “an antibody,” is understood to represent one or more antibodies. As such, the terms “a” (or “an”), “one or more,” and “at least one” can be used interchangeably herein.

The recitation of numerical ranges by endpoints includes all numbers and fractions subsumed within the respective ranges, as well as the recited endpoints.

As used herein, with “at least” a particular value means that particular value or more. For example, “at least 2” is understood to be the same as “2 or more” i.e., 2, 3, 4, 5, 6, 7, 8, 9, 10, 11, 12, 13, 14, 15, . . . , etc.

Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments described herein are capable of operation in other sequences than described or illustrated herein.

The word “about” or “approximately” when used in association with a numerical value (e.g. about 10) preferably means that the value may be the given value (of 10) more or less 5%, preferably more or less 1% of the value.

As used herein, the term “and/or” indicates that one or more of the stated cases may occur, alone or in combination with at least one of the stated cases, up to with all of the stated cases.

Various embodiments are described herein. Each embodiment as identified herein may be combined together unless otherwise indicated. Titles, subtitles, headings and the likes are used herein solely for ease of reading and are not intended to limit or restrict the disclosure in any way.

All patent applications, patents, and printed publications cited herein are incorporated herein by reference in the entireties, except for any definitions, subject matter disclaimers or disavowals, and except to the extent that the incorporated material is inconsistent with the express disclosure herein, in which case the language in this disclosure controls.

One skilled in the art will recognize many methods and materials similar or equivalent to those described herein, which could be used in the practice of the present disclosure. Indeed, the present disclosure is in no way limited to the methods and materials described. It is also understood that the disclosure encompasses the generalization of aspects of the following examples to the preceding disclosure.

The present disclosure is further described by the following examples which should not be construed as limiting the scope.

### DESCRIPTION OF THE FIGURES

FIG. 1. Schematic representations of bispecific antibody expression cassettes pINT177, a dual CMV promoter expression cassette for surface expression. pINT178, a dual promoter expression cassette for surface expression. Expression of each antibody is provided by different promoter, namely CMV and pEF.

pINT179, a dual CMV promoter surface expression cassette similar to pINT177, but where scFv is attached to the “hole” part and VHH is attached to the “knob” part of a bispecific molecule.

FIG. 2. Non-reduced Western blot of bispecific antibodies described in Table 1. Arrows indicate positions of heterodimers. Western blots were probed with anti-Fc (a) and anti-Myc (b) detection antibodies.

FIG. 3. Expression of monospecific (A, B) and bispecific (C, D) antibodies on cell surface. Cells were stained with either with human anti-Fc-phycoerythrin antibody (A, C) or anti-Myc-FITC antibody (B, D).

FIG. 4. Expression of monospecific (B, C) and bispecific (D) antibodies on cell surface. Cells were stained with anti-Flag-FITC antibody, non-transfected HEK293F cell are shown for comparison (A).

FIG. 5. Antibody binding of antigen on cell surface. Cells expressing monospecific (B, C) or bispecific (D) antibodies were incubated with 1 nM biotinylated antigen 1 and streptavidin-APC. Non-transfected HEK293F cell are shown for comparison (A).

FIG. 6. Antibody binding of antigen on cell surface. Cells expressing monospecific (B, C) or bispecific (D) antibodies were incubated with 59 nM antigen 2-FITC labelled antigen. Non-transfected HEK293F cell are shown for comparison (A).

FIG. 7. Dual antigen binding to bispecific antibodies expressed on cell surface. Cells were transfected with expressing cassettes 3-6 listed in the Table Z where antibody genes are expressed either under dual CMV promoter (A, B) or CMV-pEF promoter (C, D). Antibodies were incubated with 59 nM antigen 2-FITC labelled antigen, and 1 nM biotinylated antigen 1. Streptavidin-APC was used for detection in latter case.

FIG. 8. Titration binding of antigen 1 in the presence or absence of saturating concentration of unlabeled antigen 2. Cells were transfected with pINT178-4B12 VHH-“hole” (Flag): 3G01 scFv-“knob” (Myc) construct, and stained with increasing concentration of antigen 1 detected with streptavidin-APC. Saturating concentration of antigen 2 (180 nM) was applied prior to antigen 1 staining.

FIG. 9. FACS selection of binders from bispecific mammalian display libraries. Mammalian display libraries containing VHH repertoires directly cloned from immunized llamas (A) or following 2 rounds of phage display selection on antigen 1 (B) were stained with biotinylated antigen 1 antigen (APC secondary detection) in the presence of saturating antigen 2-FITC for enrichment of bispecific molecules capable of simultaneous binding to both antigens. Boxes indicate sort gates.

FIG. 10. Identification and confirmation of monoclonal binders from Mammalian Display bispecific library selections. Outputs form FACS selection were expressed as individual clones and bispecific antibodies were tested for antigen 1 binding by DELFIA TRF assay. Signal against antigen 1-biotin immobilized on streptavidin shows as grey bars and control values on stredtavidin only antigen (i.e. no antigen 1) are overlayed as black bars.

FIG. 11. Schematic overview of bispecific vector knob into hole technology and of the vector region between the homology arms.

### EXAMPLES

The exact antigenic targets in the below examples are not disclosed and are denoted as “antigen 1” and “antigen 2”. Both antigen 1 and antigen 2 are representative transmembrane proteins expressed by certain immune cells.

### Example 1: Construction of Vectors for Expression of Bispecific Antibodies

To effect genetic selections of binders (e.g., antibody, protein, or peptide) it is necessary to introduce a gene encoding this binder and to drive expression of this gene from either exogenous or endogenous promoter. Antibodies represent the most commonly used class of binders, and they can be formatted for expression in different forms. In examples below, we describe expression of a dual gene format where variable binding entities of different classes (e.g. VHH and scFv) are fused to the same Fc domain. In this and further examples a VHH denoted as 4B12 is an antibody that specifically binds to antigen 1, and scFvs denoted as 4F07 and 3G01 are antibodies (fragments) that specifically bind to antigen 2.

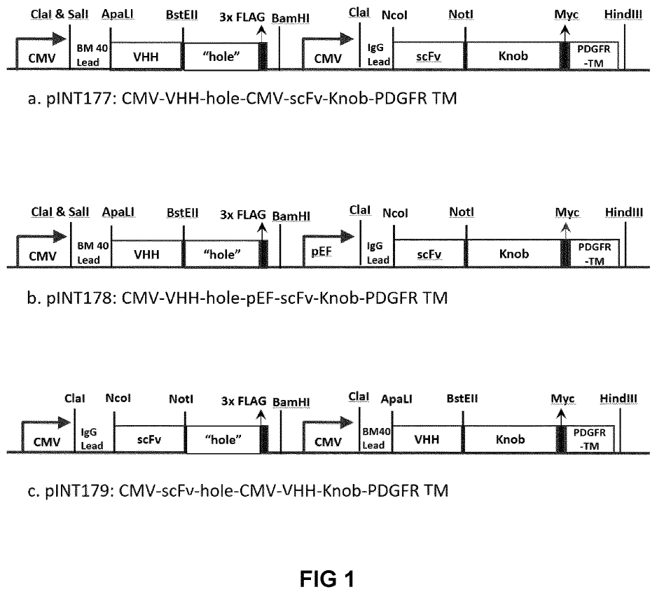

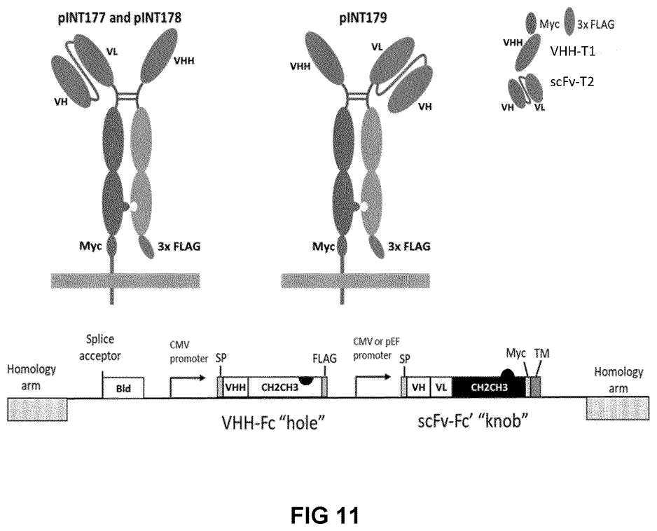

To express these bispecific antibodies in producer cells such as higher eukaryotes, it is necessary to express the separate monospecific binding polypeptide chains and facilitate correct pairing of these chains. This can be done by introducing separate plasmids encoding each chain or by introducing them on a single plasmid using multiple promoters. FIGS. 1a-c and FIG. 11 show the organisation of similar expression cassettes within the same vector backbone which were developed for expression of membrane anchored bispecific antibodies. These expression cassettes were created using a combination of gene synthesis, restriction enzyme digest and polymerase chain reaction amplification of standard elements such as promoters, antibiotic resistance genes and poly A sequences. The first plasmid was created to combine the promoter of cytomegalovirus (CMV promoter) that was used to drive expression of VHH antibody and constant regions of heavy chain containing “hole” mutations (T366S: L368A: Y407V; see also P Carter, Bispecific human IgG by design. J Immunol Methods 248, 7-15 (2001)), and for expression of scFv followed by constant regions of heavy chain containing a “knob” mutation (T366W; see also P Carter, Bispecific human IgG by design. J Immunol Methods 248, 7-15 (2001)). The latter cistron contains PDGF receptor transmembrane domain in order to provide membrane anchoring.

Secretion of the separate binding entities in the endoplasmic reticulum is directed by 2 different leader sequences. VHH secretion is directed by a BM40 leader sequence [Holden, P., Keene, D. R., Lunstrum, G. P., Bächinger, H. P., & Horton, W. A. (2005). Secretion of cartilage oligomeric matrix protein is affected by the signal peptide. J Biol Chem, 280(17), 17172-17179]. This is followed by ApaLI and BstEII restriction sites which allow in-frame cloning of any antibody. Secretion of the scFv is directed by a leader split by an intron originating from a mouse VH gene. The coding sequence of the signal peptide is followed by NcoI and NotI sites allowing in frame cloning of genes encoding the antibody with binding properties that are different from the first antibody.

In a similar way separate VHH and scFv antibodies were combined into the same backbone but with different promoters. Elongation factor-1 alpha protein is ubiquitously and abundantly expressed in most eukaryotic cells and its promoter (pEF promoter, also called the EF1alpha promoter) is commonly used for driving transgene expression [Kim, D. W., Uetsuki, T., Kaziro, Y., Yamaguchi, N., & Sugano, S. (1990). Use of the human elongation factor 1 alpha promoter as a versatile and efficient expression system. Gene, 91(2), 217-223]. In pINT178 the pEF promoter is used to drive scFv antibody expression. The polyadenylation sites originating in bovine growth hormone (BGH polyA) is present at the end of each expression cassette.

### Example 2: Western Blot of Bispecific Antibodies Expressed in Mammalian Cells

Western blot was performed to confirm bispecific antibody formation in cells transfected with the bispecific antibody display vectors. HEK293 cells were transfected with vectors listed in Table 1, using nuclease (TALEN) directed integration using homology arms targeting the NLN locus (see also Example 3 of WO2023/025834 and Examples 6 and 7 of WO2015/166272, both incorporated herein by reference. Protein fractions were prepared using RIPA buffer (ThermoFisherScientific, Cat. N 89901) as recommended by the manufacturer. Protein samples comprising antibodies were resolved by gel electrophoresis in non-reducing conditions and then transferred to PVDF membrane. The separated proteins on the membrane were then probed with anti-Fc and anti-Myc antibodies conjugated to horseradish peroxidase (HRP), and detected with HRP enhanced chemiluminescence substrate.

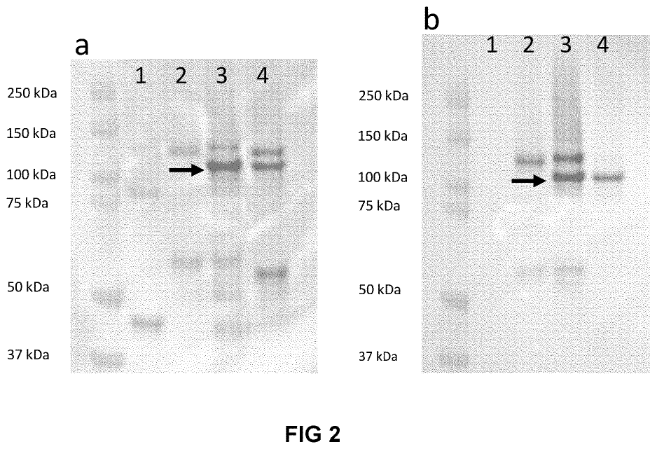

Anti-Fc and anti-Myc detection of soluble and membrane-anchored antibodies under non-reducing conditions indicates that pINT178 mostly contains heterodimers (FIG. 2, a, b, lane 3). At the same time, pINT179 (Ibid., lane 4) shows higher concentration of soluble scFv-“hole” homodimers but no detectable VHH anchored homodimers. Therefore, both vectors are capable to produce heterodimers anchored on cell surface making possible construction of bispecific mammalian display libraries.

TABLE 1
Vectors used for the transfection of the HEK293 cells

| N | Vector | Description |
|---|---|---|
| 1. | pINT172-VHH03-“hole” (Flag)-only | Monospecific, soluble |
| 2. | pINT175-FGFR1 scFv-“knob” (Myc)-only | Monospecific, anchored |
| 3. | pINT178-VHH03-“hole” (Flag): FGFR1 scFv- | Bispecific, anchored |
| | “knob”(Myc) | |
| 4. | pINT179-FGFR1scFv-“hole” (Flag): VHH03- | Bispecific, anchored |
| | “knob”(Myc) | |

### Example 3: Bispecific Antibody Expression and Antigen Binding by Flow Cytometry

To determine whether bispecific antibody expression had occurred on the cell surface, cells were transfected with either monospecific or bispecific vectors listed in the Table 2. 24 hours after transfection the volume of the bulk culture was doubled and 24 hours later blasticidin (7.5 μg/ml) was added. Cells were cultivated with the same blasticidin concentration, and medium was refreshed every 3-4 days.

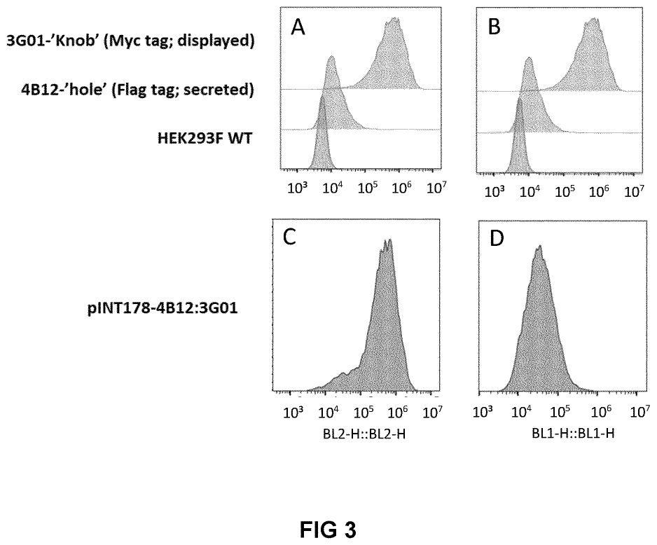

Analysis of display level for bispecific construct was carried out 15 days post-transfection using only 2.5×10\hat{ }5 cells/sample and with 0.1 mL incubation volume (following a protocol as described in Example 3 of WO2023/025834 and Examples 6 and 7 of WO2015/166272). FIG. 3 shows that at 15 days post-transfection cells express bispecific 4B12:3G01 antibody on the cell surface and this can be detected using phycoerythrin-labelled anti-Fc antibodies. Binding of anti-Myc FITC-labelled antibody to bispecific 4B12:3G01 antibody is lower than to the monospecific dual anchored 3G01 antibody containing two Myc-tags implying that former antibody construct presents heterodimer containing only one Myc-tag.

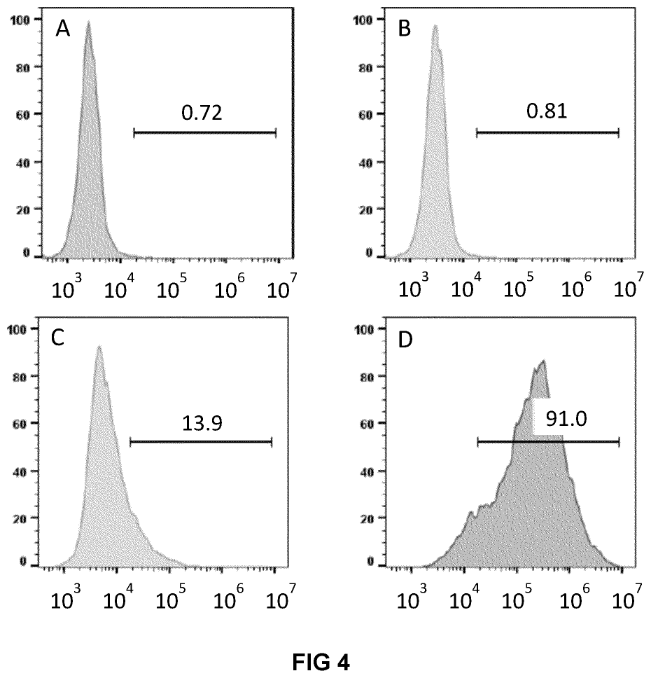

Next, in order to further validate heterodimer formation on the cell surface, staining with anti-Flag FITC-labelled antibody was carried out. Positive staining shown on FIG. 4 confirms that non-anchored bispecific antibody arm bearing Flag-tag is interacting with anchored arm through knob-in-hole mechanism resulting in bispecific heterodimer formation on the cell surface. Taken together with aforementioned findings this demonstrates expression of the bispecific antibody.

At 15 days after transfection cells were labelled with antigen 1 according to the protocol below:
1. Harvest, wash and adjust cells in 2.5×10⁶ cells per sample. Spin down cells at 250 g for 4 min, RT, wash cells with 1 ml PBS+0.1% BSA (4° C.), spin down cells at 250 g for 4 min, RT, resuspend in 1 ml PBS+1% BSA
2. Add biotinylated antigen to a final concentration 1 nM and incubate 30 min at 4° C.
3. Wash the cells 2 times 1 ml of 0.1% BSA by centrifugation at 250 g for 5 min
4. Add 1 μl of APC labelled streptavidin (ThermoScientific, Cat. N 21629) in 0.1 ml PBS+1% BSA, for 15 at 4° C. in the dark
5. Wash the cells 2 times 1 ml of 0.1% BSA by centrifugation at 250 g for 5 min
6. Resuspend them in 55 μl ice cold PBS+0.1% BSA
7. Add 7-AAD (eBioscience, Cat. N 00-6993-50) at 1:50 dilution for viability staining

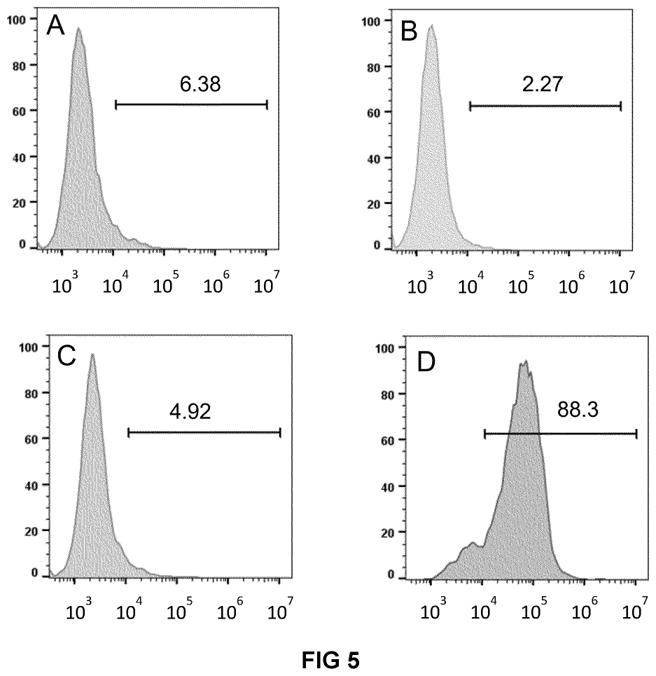

Results are shown in FIG. 5. Flow cytometry detection of the labelled antigen 1 that is bound to non-anchored arm clearly shows that heterodimer formed on the cell surface retains binding properties of VHH arm at the antigen concentration as low as 1 nM.

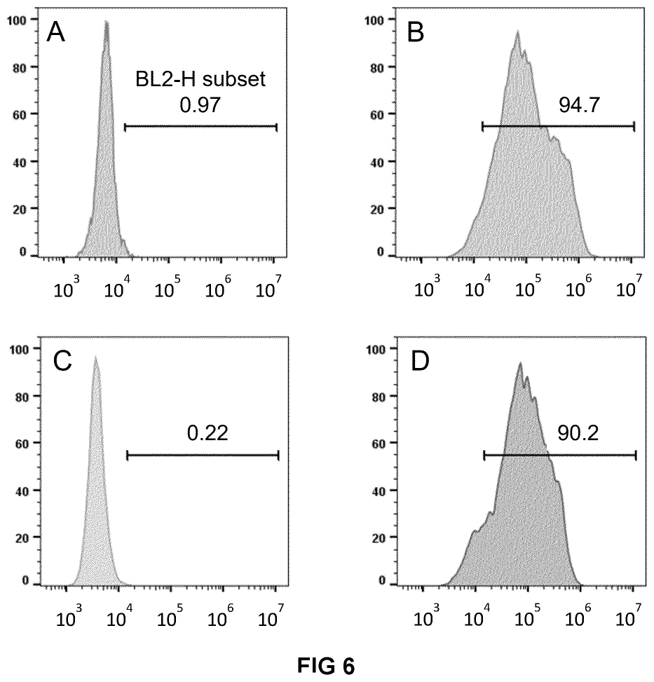

Next, similar experiment was performed to study binding of the scFv anchored arm to antigen 2 labelled with FITC. Binding of the labelled antigen 2 to scFv was detected for both bispecific and monospecific antibodies as shown on the FIG. 6.

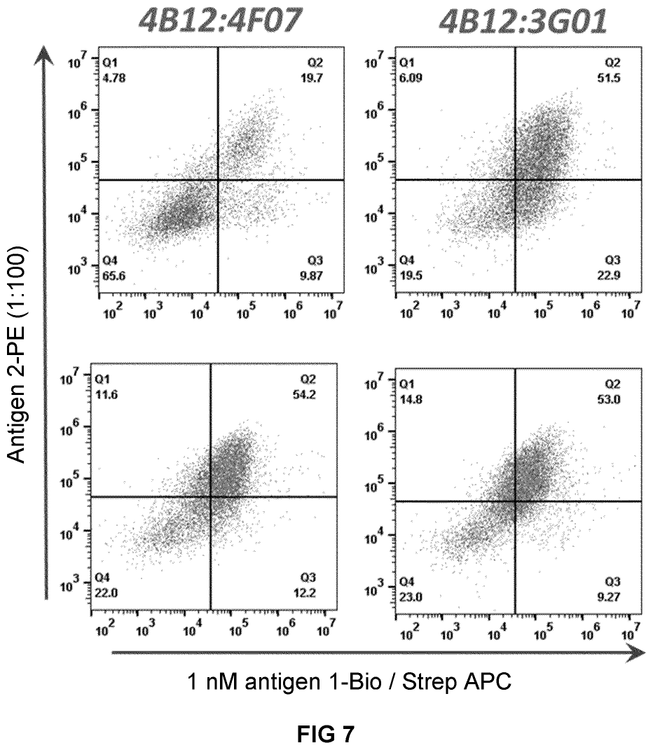

Dual antigen binding to bispecific antibodies expressed under either dual CMV or CMV-pEF promoters is shown in FIG. 7. Briefly, cells expressing 4B12 VHH and 4F07 scFv or 4B12 VHH and 3G01 scFv were stained with both antigens 1 and 2 essentially as described above. Dual staining shows detection of the bispecific antibodies irrespectively of the promoters used for expression of separate arms. Best antigen binding and bispecific display for 4B12:4F07 is observed with the pCMV pEF promoter combination.

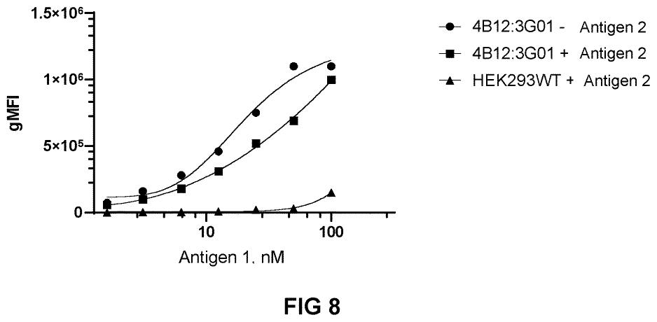

Cell lines stained with both antigens revealed presence of double-positive population. However, concentrations of the antigens were not saturating therefore it is unclear whether the bispecific antibodies bind one antigen at a time or both simultaneously. To this end, antigen 1 titration was performed in the presence and absence of saturating unlabeled antigen 2 according to the protocol outlined above. For a bispecific antibody capable of simultaneous antigen binding, similar results would be anticipated between the two conditions, whereas an antibody not capable of simultaneous binding may exhibit reduced antigen 1 binding VHH arm due to high scFv arm occupancy with antigen 2. Data presented on FIG. 8 shows little if any competition for antigen 1 binding demonstrating simultaneously occupancy of both binding arms with respective antigens.

TABLE 2
Vectors used for the transfection of the HEK293 cells

| N | Vector | Description |
|---|---|---|
| 1. | pINT172-4B12 VHH-“hole” (Flag)-only | Monospecific, soluble |
| 2. | pINT175-3G01 scFv-“knob” (Myc)-only | Monospecific, anchored |
| 3. | pINT178-4B12 VHH-“hole” (Flag): 3G01 scFv- | Bispecific, anchored |
| | “knob”(Myc) | |
| 4. | pINT178-4B12 VHH-“hole” (Flag): 4F07 scFv- | Bispecific, anchored |
| | “knob”(Myc) | |
| 5. | pINT177-4B12 VHH-“hole” (Flag): 3G01 scFv- | Bispecific, anchored |
| | “knob”(Myc) | |
| 6. | pINT177-4B12 VHH-“hole” (Flag): 4F07 scFv- | Bispecific, anchored |
| | “knob”(Myc) | |

### Example 4: Identification of Novel Bispecific Antibody Binders from Mammalian Display Libraries

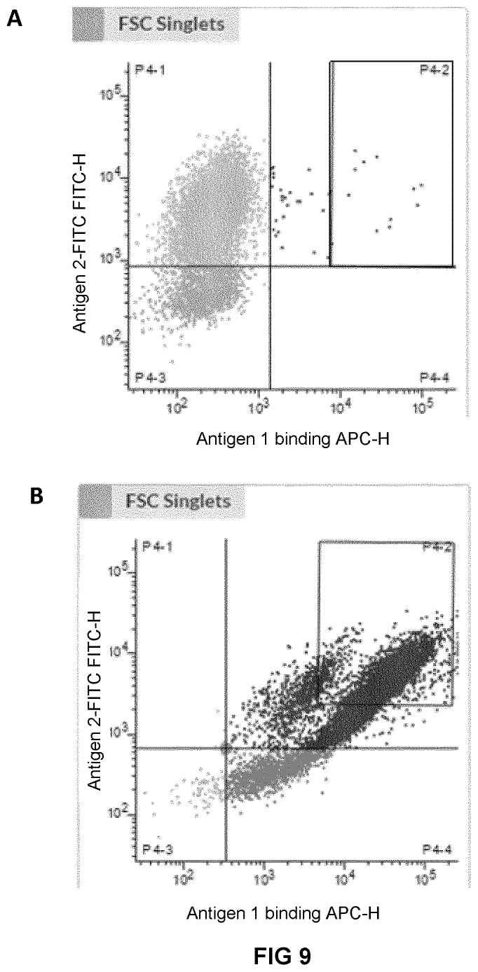

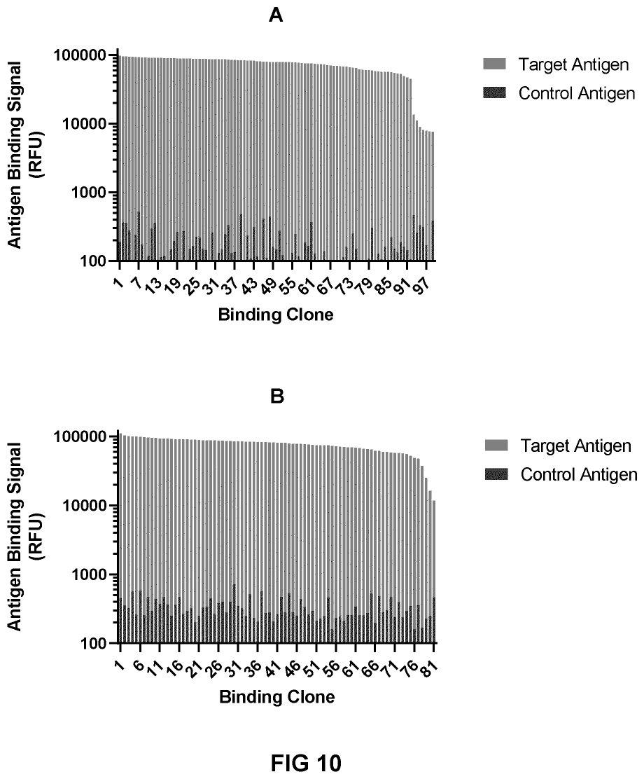

To validate use of the system to select monoclonal binders from libraries of bispecific molecules displayed on the surface of mammalian cells, anti-antigen 1 VHH repertoires directly from immunized llamas, or following 2 rounds of phage display, were cloned into the pINT178 vector system by restriction cloning using ApaL1 and BstEII. Mammalian display libraries of bispecific molecules were generated from the resulting pINT178 plasmid libraries as described in detail previously (WO2015/166272, WO2023/025834, Parthiban et al. MAbs 2019; 11(5); 884-898; Dyson et al. MAbs 2020; 12(1): 1829335). Briefly, HEK293 cells were transfected unsing the MaxCyte electroporation system, and passaged with antibiotic selection (7.5 μg/ml blasticidin). Magnetic-activated cell sorting (MACS) for antibody expression was performed at least 6 days post-transfection to generate integrated mammalian display libraries of 10⁵-10⁷ clones. FACS selections from these bispecific mammalian display libraries are shown in FIG. 9 (FACS of the library directly cloned from immunized llamas is presented in panel A and the phage display enriched input library is shown in panel B). Cell staining was performed as described previously and above (Parthiban et al. MAbs 2019; WO2015/166272). Cells were sorted for antibody binding to biotinylated antigen 1 (allophycocyanin (APC) secondary detection) in the presence of saturating antigen 2-FITC (sort gates represented as boxes in FIG. 9) for enrichment of bispecific molecules capable of simultaneous binding to both antigens. As expected, antigen 2 binding was retained as a fixed modality in the bispecific constructs (y-axis) and antigen 1 binding (x-axis) is enriched in the phage display input relative to the directly cloned immune library. Low frequency VHH antigen 1 binders that are compatible with the bispecific format and antigen 2 occupancy in the scFv-Fc arm are clearly detectable in the llama immune repertoire. Enriched bispecific antibody clones were isolated and screened for binding to antigen 1 using a DELFIA TRF assay (FIG. 10). DELFIA® (dissociation-enhanced lanthanide fluorescence immunoassay) is a time-resolved fluorescence (TRF) intensity technology (Perkin Elmer) commonly used in the industry as alternative binding assay to ELISA. 97 monoclonal binders were identified directly from the immunised repertoire and 81 from the phage enriched library. Taken together, Examples 1 to 4 demonstrate display of bispecific antibody libraries on mammalian cells and their use to identify novel bispecific molecules of interest.

### Overview of Sequences

| SEQ ID NO | Description of the sequence |
|---|---|
| 1 | I-Scel recognition sequence |
| 2 | Conserved structural motif |
| 3 | NLN gene |
| 4 | TNIK gene |
| 5 | PARP 11 gene |
| 6 | RAB40B gene |
| 7 | ABI2 gene |
| 8 | RNF19B gene |
| 9 | PKIA gene |
| 10 | FTCD gene |
| 11 | NLN-201 intron 1 |
| 12 | NLN-201 intron 2 |
| 13 | NLN-201 intron 3 |
| 14 | NLN-201 intron 4 |
| 15 | NLN-201 intron 5 |
| 16 | NLN-201 intron 6 |
| 17 | NLN-201 intron 7 |
| 18 | NLN-201 intron 8 |
| 19 | NLN-201 intron 9 |
| 20 | NLN-201 intron 10 |
| 21 | NLN-201 intron 11 |
| 22 | NLN-201 intron 12 |
| 23 | NLN-207 intron 5 |
| 24 | NLN-207 intron 6 |
| 25 | GGGS flexible linker unit |

1. A method for producing a library of eukaryotic cell clones containing DNA encoding a diverse repertoire of bispecific binders, the method comprising:
providing eukaryotic cells;
providing a plurality of donor DNA molecules, each donor DNA molecule comprising a first nucleic acid sequence encoding a first binding domain coupled to a first Fc domain; and a second nucleic acid sequence encoding a second binding domain coupled to a second Fc domain, wherein the first and second Fc domains are engineered to promote heterodimerization;
introducing the donor DNA into the cells; and
culturing the recombinant cells to produce clones, thereby providing a library of eukaryotic cell clones containing DNA encoding the repertoire of bispecific binders.

2. A method according to claim 1, wherein the first and/or second binding domain comprises a single antibody variable domain, preferably a variable domain derived from a heavy-chain antibody such as a VHH or a V_{NAR}, and/or wherein the first and/or second binding domain comprises two antibody variable domains, preferably a single-chain variable fragment (scFv).

3. A method according to claim 1, wherein the first binding domain comprises a single antibody variable domain, preferably a variable domain derived from a heavy-chain antibody such as a VHH or a V_{NAR}, more preferably a VHH.

4. A method according to claim 1, wherein the second binding domain comprises two antibody variable domains, preferably a single-chain variable fragment (scFv).

5. A method according to claim 1, wherein the first Fc domain comprises a knob mutation and the second Fc domain comprises a hole mutation, or wherein the first Fc domain comprises a hole mutation and the second Fc domain comprises a knob mutation.

6. A method according to claim 1, wherein the step of introducing the donor DNA into the cells comprises providing a site-specific nuclease within the cells, wherein the nuclease cleaves a recognition sequence in cellular DNA to create an integration site at which the donor DNA becomes integrated into the cellular DNA, integration occurring through DNA repair mechanisms endogenous to the cells, thereby creating recombinant cells containing donor DNA integrated in the cellular DNA.

7. A method according to claim 1, wherein the donor DNA molecules are flanked by homology arms.

8. A method according to claim 1, wherein the donor DNA molecules comprise a first promoter operably linked to the first nucleic acid sequence and/or wherein the donor DNA molecules comprise a second promoter operably linked to the second nucleic acid sequence.

9. A method according to claim 1, wherein the donor DNA molecules comprise a bidirectional promoter operably linked to the first and second nucleic acid sequence.

10. A method according to claim 1, wherein the first and/or second nucleic acid sequence encodes a membrane anchor, such as a transmembrane domain or a membrane localization signal.

11. A method according to claim 1, wherein the eukaryotic cells are higher eukaryotic cells with a genome size of greater than 2×10⁷ base pairs, preferably wherein the cells are mammalian, avian, insect or plant cells, more preferably wherein the cells are mammalian cells.

12. A method of screening for a bispecific binder that recognises a target of interest, comprising:
producing a library by the method of claim 1;
culturing cells of the library to express the bispecific binders;
exposing the bispecific binders to the target, optionally the two targets, allowing recognition of the target, optionally of the two targets, by one or more binders of interest, if present; and
detecting whether the target, optionally the two targets, is recognised by a binder of interest, optionally further comprising recovering cells of a clone containing DNA encoding the binder of interest, optionally further comprising isolating DNA encoding the binder from the recovered clone.

13. A method of screening for a cell of a desired phenotype, wherein the phenotype results from expression of a bispecific binder by the cell, comprising:
producing a library by the method of claim 1;
culturing cells of the library to express the bispecific binders; and
detecting whether the desired phenotype is exhibited,
optionally further comprising recovering cells of a clone that expresses a binder that produced the desired phenotype,
optionally further comprising isolating DNA encoding the binder from the recovered clone.

14. A library produced by a method according to claim 1.

15. An in vitro library of eukaryotic cell clones that express a diverse repertoire of at least 10\hat{ }3, 10\hat{ }4, 10\hat{ }5, 10\hat{ }6, 10\hat{ }7, 10\hat{ }8 or 10\hat{ }9 different bispecific binders, each cell containing recombinant DNA wherein donor DNA encoding a bispecific binder or subunit of a bispecific binder is integrated in at least a first and/or a second locus in the cellular DNA; optionally wherein the locus is a fixed locus.
